(19) United States
(12) Patent Application Publication
(10) Pub. No.: US 2024/0398510 A1
(43) Pub. Date: Dec. 5, 2024

(54) REMOTE ORTHODONTIC PROGRESS TRACKING USING PHYSICAL BITE IMPRESSIONS

(71) Applicant: ALIGN TECHNOLOGY, INC., San Jose, CA (US)

(72) Inventors: Yosi MOALEM, Ness-Ziona (IL); Ofer SAPHIER, Rehovot (IL)

(73) Assignee: ALIGN TECHNOLOGY, INC., San Jose, CA (US)

(21) Appl. No.: 18/679,264

(22) Filed: May 30, 2024

Related U.S. Application Data
(60) Provisional application No. 63/505,331, filed on May 31, 2023.

(51) Int. Cl. A61C 7/00; A61C 13/34; G06T 19/20; G16H 40/67

(52) U.S. Cl. CPC A61C 7/002; A61C 13/34; G06T 19/20; G16H 40/67; G06T 2210/41; G06T 2219/2004

(57) ABSTRACT

Remote progress tracking of orthodontic treatment may include receiving a 3D digital model of a patient's dentition including the patient's teeth. A 3D digital model of an impression of occlusal surfaces of the patient's teeth may be generated. The teeth of the 3D digital model of the patient's dentition may be registered to the position and orientation of the patient's teeth in the 3D digital model of the impression of the occlusal surfaces of the patient's teeth. An updated 3D digital model of the patient's teeth may be generated based on the registration.

### RELATED APPLICATIONS

This application claims the benefit under 35 U.S.C. § 119 (e) of U.S. Provisional Patent Application No. 63/505,331, filed May 31, 2023, and titled “SYSTEMS AND METHODS FOR REMOTE ORTHODONTIC PROGRESS TRACKING USING WAX BITE IMPRESSIONS,” which is incorporated, in its entirety, by this reference.

### BACKGROUND

Orthodontic treatment with aligners can be a highly effective way to straighten teeth and correct bite issues. However, in some cases, the treatment can go off track, which can lead to delays in treatment and less-than-optimal results. There are several reasons why aligner treatment may go off track, including patient noncompliance, problems with the aligners themselves, and unexpected changes in the teeth or gums.

If an orthodontic treatment goes off track, the first step is to schedule an in-person consultation with the orthodontist. During the consultation, the orthodontist examines the patient's teeth and assess the progress of the treatment. Depending on the nature of the problem, the orthodontist may recommend several different courses of action.

If there is a problem with the aligners themselves, such as a crack or a misshapen tray, the orthodontist may recommend ordering a new set of aligners to replace the problematic ones. In some cases, it may be necessary to take new impressions of the teeth to ensure that the new aligners fit correctly.

These current processes are less than ideal for a number of reasons. For example, an in-person consultation involves scheduling the appointment in advance, which further delays treatment and result in further off-track treatment. The in-person consultation also usually includes the use of an intraoral scanner to generate a new 3D model of the patient's dentition and further processing.

In light of the above, improved devices and methods that overcome at least some of the above limitations of the prior devices and methods would be helpful.

### SUMMARY

Embodiments of the present disclosure provide improved off-track treatment systems and methods that provide accurate models of the patient's dentition for further evaluation and corrective orthodontic treatment.

### BRIEF DESCRIPTION OF THE DRAWINGS

A better understanding of the features, advantages and principles of the present disclosure will be obtained by reference to the following detailed description that sets forth illustrative embodiments, and the accompanying drawings of which:

FIG. 1A illustrates an exemplary tooth repositioning appliance or aligner that can be worn by a patient in order to achieve an incremental repositioning of individual teeth in the jaw, in accordance with some embodiments herein;

FIG. 1B illustrates a tooth repositioning system, in accordance with some embodiments herein;

FIG. 2 shows a method of orthodontic treatment using a plurality of appliances, in accordance with some embodiments herein;

FIG. 3 shows a method for digitally planning an orthodontic treatment, in accordance with some embodiments herein;

FIG. 4 shows a simplified block diagram of a data processing system, in accordance with some embodiments herein;

FIG. 5 shows bite blocks, in accordance with some embodiments herein;

FIG. 6 shows an impression in a bite block, in accordance with some embodiments herein;

FIG. 7 shows view of a 3D model of the patient's dentition, in accordance with some embodiments herein;

FIG. 8 shows a view of a 3D model of the patient's dentition aligned with a 3D model of an impression, in accordance with some embodiments herein;

FIG. 9 shows views of a 3D model of the patient's dentition with tooth positions based on a 3D model of an impression, in accordance with some embodiments herein;

FIG. 10 shows a method of generating an updated treatment plan based on tooth positions from an impression, in accordance with some embodiments herein;

FIG. 11 shows a method of generating an updated retainer based on tooth positions from an impression, in accordance with some embodiments herein;

FIG. 12 shows a computing system, in accordance with some embodiments herein; and

FIG. 13 shows a network architecture, in accordance with some embodiments herein.

FIG. 14 shows a method of updating a treatment plan, in accordance with some embodiments herein.

FIG. 15 shows bite blocks, in accordance with some embodiments herein.

FIG. 16 shows aspects of a method of scanning bite blocks, in accordance with some embodiments herein.

FIG. 17 shows aspects of an apparatus for scanning bite blocks, in accordance with some embodiments herein.

FIG. 18 shows aspects of an apparatus for scanning bite blocks, in accordance with some embodiments herein.

### DETAILED DESCRIPTION

The following detailed description provides a better understanding of the features and advantages of the inventions described in the present disclosure in accordance with the embodiments disclosed herein. Although the detailed description includes many specific embodiments, these are provided by way of example only and should not be construed as limiting the scope of the inventions disclosed herein.

The methods, apparatus, and systems disclosed herein are well suited for combination with prior orthodontic treatment systems and processes, for example the Invisalign system commercially available from Align Technology, Inc.

Orthodontic treatment with aligners uses a series of clear plastic aligners to gradually move teeth from an initial arrangement towards a desired arrangement in a series of stages. These aligners are designed to fit over the teeth and being clear, they are hard to see, making them a popular option for patients who are looking for a discreet way to straighten their teeth.

The aligner treatment process begins with an initial consultation with an orthodontist. During this consultation, the orthodontist performs a comprehensive evaluation of the patient's teeth, gums, and jaw to determine if aligner treatment is the right option for them. If so, the orthodontist may use an intraoral 3D scanner to scan the patient's intraoral cavity and create a 3D digital model of the patient's teeth.

A treatment plan may be generated based on the 3D model. The treatment plan is a series of stages. Each stage defines the movements and/or forces used to move the teeth one incremental step towards the final positions (which may also be referred to as target positions) and the associated aligner shapes for moving the teeth during the stage of treatment.

Once the treatment plan is developed, the aligners are fabricated from a durable, clear polymer material. When the aligners are ready, the patient return to the orthodontist to have the first set of aligners fitted.

The aligners are worn for a minimum of 20-22 hours per day, but can be removed for eating, brushing, and flossing. Patients typically wear each set of aligners (a set including an aligner of one or both of the upper and lower arches of the patient's dentition) for two weeks before moving on to the next set in the series. Each set of aligners will gradually move the teeth closer to their desired positions, until the final set is reached and the treatment is complete. Orthodontic treatment using aligners may involve wearing a series of 10 to 30 aligners for 4 to 18 months or more, depending on the complexity of the patient's case.

Orthodontic treatment with aligners can be an effective option for a variety of orthodontic issues, including crowded teeth, gaps between teeth, overbites, underbites, crossbites, and other malocclusions of the patient's dentition.

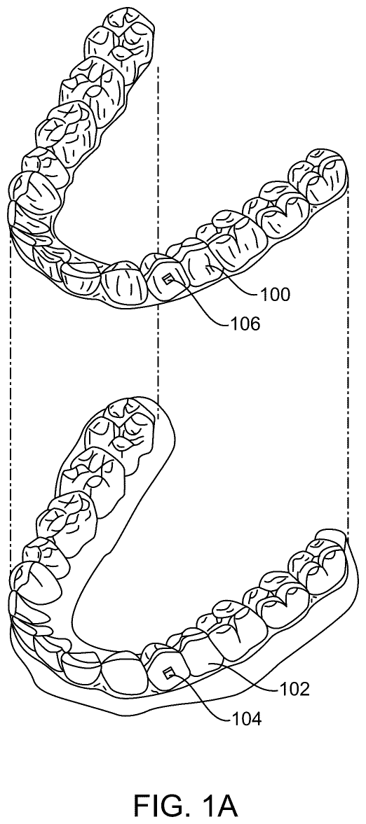

FIG. 1A illustrates an exemplary tooth repositioning appliance 100, such as an aligner that can be worn by a patient in order to achieve an incremental repositioning of individual teeth 102 in the jaw. The appliance can include a shell (e.g., a continuous polymeric shell or a segmented shell) having teeth-receiving cavities that receive and resiliently reposition the teeth. An appliance or portion(s) thereof may be indirectly fabricated using a physical model of teeth. For example, an appliance (e.g., polymeric appliance) can be formed using a physical model of teeth and a sheet of suitable layers of polymeric material. The physical model (e.g., physical mold) of teeth can be formed through a variety of techniques, including 3D printing. The appliance can be formed by thermoforming the appliance over the physical model. In some embodiments, a physical appliance is directly fabricated, e.g., using additive manufacturing techniques, from a digital model of an appliance. In some embodiments, the physical appliance may be created through a variety of direct formation techniques, such as 3D printing. An appliance can fit over all teeth present in an upper or lower jaw, or less than all of the teeth. The appliance can be designed specifically to accommodate the teeth of the patient (e.g., the topography of the tooth-receiving cavities matches the topography of the patient's teeth) and may be fabricated based on positive or negative models of the patient's teeth generated by impression, scanning, and the like. Alternatively, the appliance can be a generic appliance configured to receive the teeth, but not necessarily shaped to match the topography of the patient's teeth. In some cases, only certain teeth received by an appliance will be repositioned by the appliance while other teeth can provide a base or anchor region for holding the appliance in place as it applies force against the tooth or teeth targeted for repositioning. In some cases, some or most, and even all, of the teeth will be repositioned at some point during treatment. Teeth that are moved can also serve as a base or anchor for holding the appliance as it is worn by the patient. In some embodiments, no wires or other means will be provided for holding an appliance in place over the teeth. In some cases, however, it may be desirable or necessary to provide individual attachments or other anchoring elements 104 on teeth 102 with corresponding receptacles or apertures 106 in the appliance 100 so that the appliance can apply a selected force on the tooth. Exemplary appliances, including those utilized in the Invisalign® System, are described in numerous patents and patent applications assigned to Align Technology, Inc. including, for example, in U.S. Pat. Nos. 6,450,807, and 5,975,893, as well as on the company's website, which is accessible on the World Wide Web (see, e.g., the URL “invisalign.com”). Examples of tooth-mounted attachments suitable for use with orthodontic appliances are also described in patents and patent applications assigned to Align Technology, Inc., including, for example, U.S. Pat. Nos. 6,309,215 and 6,830,450.

FIG. 1B illustrates a tooth repositioning system 101 including a plurality of appliances 103A, 103B, 103C. Any of the appliances described herein can be designed and/or provided as part of a set of a plurality of appliances used in a tooth repositioning system. Each appliance may be configured so a tooth-receiving cavity has a geometry corresponding to an intermediate or final tooth arrangement intended for the appliance. The patient's teeth can be progressively repositioned from an initial tooth arrangement to a target tooth arrangement by placing a series of incremental position adjustment appliances over the patient's teeth. For example, the tooth repositioning system 101 can include a first appliance 103A corresponding to an initial tooth arrangement, one or more intermediate appliances 103B corresponding to one or more intermediate arrangements, and a final appliance 103C corresponding to a target arrangement. A target tooth arrangement can be a planned final tooth arrangement selected for the patient's teeth at the end of all planned orthodontic treatment. Alternatively, a target arrangement can be one of some intermediate arrangements for the patient's teeth during the course of orthodontic treatment, which may include various different treatment scenarios, including, but not limited to, instances where surgery is recommended, where interproximal reduction (IPR) is appropriate, where a progress check is scheduled, where anchor placement is best, where palatal expansion is desirable, where restorative dentistry is involved (e.g., inlays, onlays, crowns, bridges, implants, veneers, and the like), etc. As such, it is understood that a target tooth arrangement can be any planned resulting arrangement for the patient's teeth that follows one or more incremental repositioning stages. Likewise, an initial tooth arrangement can be any initial arrangement for the patient's teeth that is followed by one or more incremental repositioning stages.

Optionally, in cases involving more complex movements or treatment plans, it may be beneficial to utilize auxiliary components (e.g., features, accessories, structures, devices, components, and the like) in conjunction with an orthodontic appliance. Examples of such accessories include but are not limited to elastics, wires, springs, bars, arch expanders, palatal expanders, twin blocks, occlusal blocks, bite ramps, mandibular advancement splints, bite plates, pontics, hooks, brackets, headgear tubes, springs, bumper tubes, palatal bars, frameworks, pin-and-tube apparatuses, buccal shields, buccinator bows, wire shields, lingual flanges and pads, lip pads or bumpers, protrusions, divots, and the like. In some embodiments, the appliances, systems and methods described herein include improved orthodontic appliances with integrally formed features that are shaped to couple to such auxiliary components, or that replace such auxiliary components.

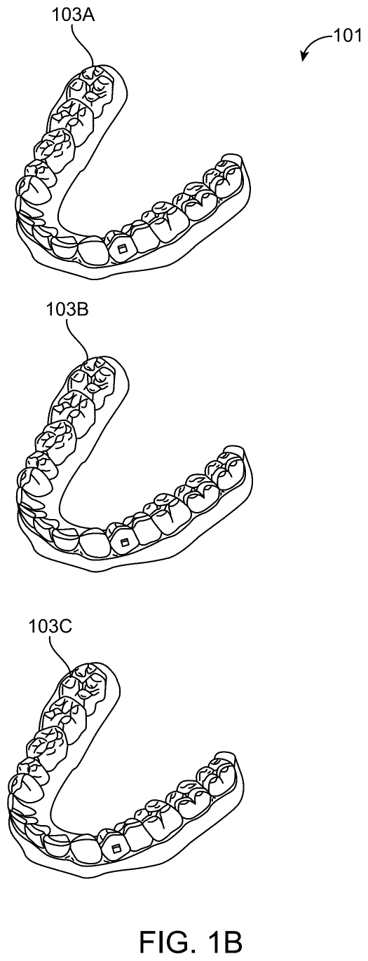

FIG. 2 illustrates a method 200 of orthodontic treatment using a plurality of appliances, in accordance with many embodiments. The method 200 can be practiced using any of the appliances or appliance sets described herein. In step 320, a first orthodontic appliance is applied to a patient's teeth in order to reposition the teeth from a first tooth arrangement to a second tooth arrangement. In step 220, a second orthodontic appliance is applied to the patient's teeth in order to reposition the teeth from the second tooth arrangement to a third tooth arrangement. The method 200 can be repeated as necessary using any suitable number and combination of sequential appliances in order to incrementally reposition the patient's teeth from an initial arrangement to a target arrangement. The appliances can be generated all at the same stage or in sets or batches (e.g., at the beginning of a stage of the treatment), or one at a time, and the patient can wear each appliance until the pressure of each appliance on the teeth can no longer be felt or until the maximum amount of expressed tooth movement for that given stage has been achieved. A plurality of different appliances (e.g., a set) can be designed and even fabricated prior to the patient wearing any appliance of the plurality. After wearing an appliance for an appropriate period of time, the patient can replace the current appliance with the next appliance in the series until no more appliances remain. The appliances are generally not affixed to the teeth and the patient may place and replace the appliances at any time during the procedure (e.g., patient-removable appliances). The final appliance or several appliances in the series may have a geometry or geometries selected to overcorrect the tooth arrangement. For instance, one or more appliances may have a geometry that would (if fully achieved) move individual teeth beyond the tooth arrangement that has been selected as the “final.” Such over-correction may be desirable in order to offset potential relapse after the repositioning method has been terminated (e.g., permit movement of individual teeth back toward their pre-corrected positions). Over-correction may also be beneficial to speed the rate of correction (e.g., an appliance with a geometry that is positioned beyond a desired intermediate or final position may shift the individual teeth toward the position at a greater rate). In such cases, the use of an appliance can be terminated before the teeth reach the positions defined by the appliance. Furthermore, over-correction may be deliberately applied in order to compensate for any inaccuracies or limitations of the appliance.

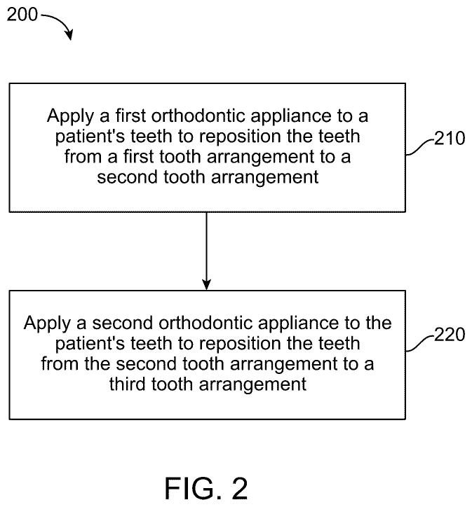

FIG. 3 illustrates a method 300 for digitally planning an orthodontic treatment and/or design or fabrication of an appliance, in accordance with many embodiments. The method 300 can be applied to any of the treatment procedures described herein and can be performed by any suitable data processing system. Any embodiment of the appliances described herein can be designed or fabricated using the method 300.

In step 310, a digital representation of a patient's teeth is received. The digital representation can include surface topography data for the patient's intraoral cavity (including teeth, gingival tissues, etc.). The surface topography data can be generated by directly scanning the intraoral cavity, a physical model (positive or negative) of the intraoral cavity, or an impression of the intraoral cavity, using a suitable scanning device (e.g., a handheld scanner, desktop scanner, etc.).

In step 320, one or more treatment stages are generated based on the digital representation of the teeth. The treatment stages can be incremental repositioning stages of an orthodontic treatment procedure designed to move one or more of the patient's teeth from an initial tooth arrangement to a target arrangement. For example, the treatment stages can be generated by determining the initial tooth arrangement indicated by the digital representation, determining a target tooth arrangement, and determining movement paths of one or more teeth in the initial arrangement necessary to achieve the target tooth arrangement. The movement path can be optimized based on minimizing the total distance moved, preventing collisions between teeth, avoiding tooth movements that are more difficult to achieve, or any other suitable criteria.

In step 330, at least one orthodontic appliance is fabricated based on the generated treatment stages. For example, a set of appliances can be fabricated to be sequentially worn by the patient to incrementally reposition the teeth from the initial arrangement to the target arrangement. Some of the appliances can be shaped to accommodate a tooth arrangement specified by one of the treatment stages. Alternatively or in combination, some of the appliances can be shaped to accommodate a tooth arrangement that is different from the target arrangement for the corresponding treatment stage. For example, as previously described herein, an appliance may have a geometry corresponding to an overcorrected tooth arrangement. Such an appliance may be used to ensure that a suitable amount of force is expressed on the teeth as they approach or attain their desired target positions for the treatment stage. As another example, an appliance can be designed in order to apply a specified force system on the teeth and may not have a geometry corresponding to any current or planned arrangement of the patient's teeth.

In some instances, staging of various arrangements or treatment stages may not be necessary for design and/or fabrication of an appliance. As illustrated by the dashed line in FIG. 3, design and/or fabrication of an orthodontic appliance, and perhaps a particular orthodontic treatment, may include use of a representation of the patient's teeth (e.g., receive a digital representation of the patient's teeth 310), followed by design and/or fabrication of an orthodontic appliance based on a representation of the patient's teeth in the arrangement represented by the received representation.

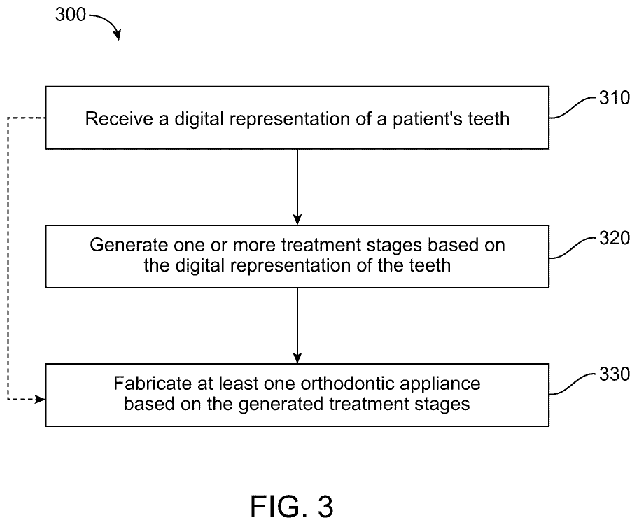

FIG. 4 is a simplified block diagram of a data processing system 1400 that may be used in executing methods and processes described herein. In some embodiments, a data processing system may be an intraoral or scanning system. The data processing system 400 typically includes at least one processor 402 that communicates with one or more peripheral devices via bus subsystem 404. These peripheral devices typically include a storage subsystem 406 (memory subsystem 408 and file storage subsystem 414), a set of user interface input and output devices 418, and an interface to outside networks 416. This interface is shown schematically as “Network Interface” block 416, and is coupled to corresponding interface devices in other data processing systems via communication network interface 424. Data processing system 400 can include, for example, one or more computers, such as a personal computer, workstation, mainframe, laptop, and the like.

The user interface input devices 418 are not limited to any particular device, and can typically include, for example, a keyboard, pointing device, mouse, scanner, interactive displays, touchpad, joysticks, etc. Similarly, various user interface output devices can be employed in a system of the invention, and can include, for example, one or more of a printer, display (e.g., visual, non-visual) system/subsystem, controller, projection device, audio output, and the like.

Storage subsystem 406 maintains the basic required programming, including computer readable media having instructions (e.g., operating instructions, etc.), and data constructs. The program modules discussed herein are typically stored in storage subsystem 406. Storage subsystem 406 typically includes memory subsystem 408 and file storage subsystem 414. Memory subsystem 408 typically includes a number of memories (e.g., RAM 410, ROM 412, etc.) including computer readable memory for storage of fixed instructions, instructions and data during program execution, basic input/output system, etc. File storage subsystem 414 provides persistent (non-volatile) storage for program and data files, and can include one or more removable or fixed drives or media, hard disk, floppy disk, CD-ROM, DVD, optical drives, and the like. One or more of the storage systems, drives, etc. may be located at a remote location, such coupled via a server on a network or via the internet/World Wide Web. In this context, the term “bus subsystem” is used generically so as to include any mechanism for letting the various components and subsystems communicate with each other as intended and can include a variety of suitable components/systems that would be known or recognized as suitable for use therein. It will be recognized that various components of the system can be, but need not necessarily be at the same physical location, but could be connected via various local-area or wide-area network media, transmission systems, etc.

Scanner 420 includes any means for obtaining a digital representation (e.g., images, surface topography data, etc.) of a patient's teeth (e.g., by scanning physical models of the teeth such as casts 421, by scanning impressions taken of the teeth, or by directly scanning the intraoral cavity), which can be obtained either from the patient or from treating professional, such as an orthodontist, and includes means of providing the digital representation to data processing system 400 for further processing. Scanner 420 may be located at a location remote with respect to other components of the system and can communicate image data and/or information to data processing system 400, for example, via a network interface 424. Fabrication system 422 fabricates appliances 423 based on a treatment plan, including data set information received from data processing system 400. Fabrication machine 422 can, for example, be located at a remote location and receive data set information from data processing system 400 via network interface 424.

While orthodontic treatment with aligners is a highly effective way to straighten teeth and correct malocclusions. In some cases, the treatment can go off track during or after treatment, which can lead to delays in completing treatment and less-than-optimal results. There are several reasons why orthodontic treatment may go off track, including patient noncompliance, problems with the aligners themselves, missed monitoring appointments, lost retainer, or lack of retainer fit due to lack of use of the retainer and unexpected changes in the teeth or gums.

If an orthodontic treatment goes off track, the first step may be to schedule an in-person consultation with the orthodontist. During the consultation, the orthodontist examines the patient's teeth and assess the progress of the treatment. Depending on the nature of the problem, the orthodontist may recommend several different courses of action.

However, an in-person consultation involves scheduling the appointment in advance, which further delays treatment and result in further off-track treatment. The in-person consultation also usually includes the use of an intraoral scanner to generate a new 3D model of the patient's dentition and further processing.

Newly developed systems and methods allows a new 3D model of the patient's dentition to be generated without a trip to the orthodontic or other dental professional. In some embodiments, the new or updated 3D model may be bested on a previously generated model, such as the model generated during the initial treatment planning phase of treatment, as described herein, and a user provided bite impression. The bite impression may be made using a dental wax sheet or bite block or other impressioning material, such as dental bite block 500.

The dental bite block 500 is a type of dental impression tool that may be made of wax or other impressioning material and is used to take an impression of the occlusal surfaces of the patient's teeth and bite. The wax bite block is typically softer and more pliable than other types of impression materials, such as silicone or alginate.

Although reference is made herein to a wax bite block, other materials may be used, such as silicone or alginate, and other forms may be used, such as wax sheet material.

The wax of a dental wax bite block can be softened at relatively low temperatures, such as using hot tap water at greater than 104 degrees F. After softening the wax bite block a patient may adjust the shape of the bite block to fit within the patient's mouth and cover the occlusal and incisal surfaces of the patient's dentition during occlusion. The patient can then bite down on the wax bite block and maintain their teeth in occlusion until the wax hardens. The wax may have a working time of between 20 and 40 seconds. The wax may harden at temperatures typically found in the mouth, such as at or below 98 degrees F. The wax after hardening is stiffer and more durable than the wax before being heated and may maintain its shape even if reheated.

The resulting bite block impression captures the shape and position of the occlusal surfaces of the patient's teeth and bite. The shape and position of the occlusal surfaces may be used to generate a full 3D model of the patient's dentition in the teeth's current arrangement based on the previously scanned 3D model, as discussed herein.

The bite block may have a thickness 504 of between 2 mm and 10 mm. In some embodiments, the bite block may have a thickness of between 3 mm and 6 mm. In some embodiments, the bite block may have a thickness of between 5 mm and 10 mm. The thickness of the bite block may be based on an expected depth to which the occlusal portions of the teeth intrude into the bite block. In some embodiments, the patient's teeth may protrude only 1 mm or less into the bite block in order to generate an acceptable depth to capture the occlusal features of the patient's teeth in a scan. In some embodiments, less than 10% of the crown height of the patient's tooth penetrates the surface of the bite block. In some embodiments, the teeth press into the bite block a depth sufficient to generate an impression of the cusps and grooves of the patient's teeth. In some embodiments, less than 10% of the surface area of the patient's tooth is compressed into the bite block.

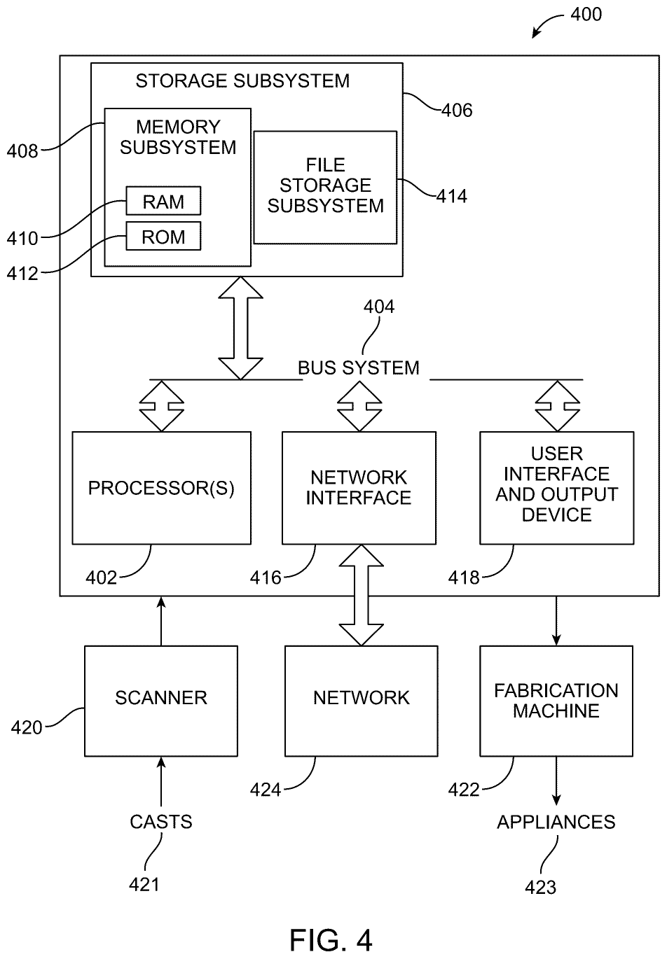

The bite block may have a pattern 502 on one or both occlusal surfaces 506 of the bite block 500. The pattern 502 may be a grid pattern, such as the grid pattern depicted on the central bite block 500 of FIG. 5. In some embodiments, the pattern 502 may be a pattern of dots. The pattern 502 may help in digitizing and scanning the impression. For example, the pattern 502 may be in a known size and shape prior to the patient biting down on the material. When the patient bites down on the block, the pattern may be deformed. By imaging the pattern after such deformation (e.g., using a 2D camera of a phone, scanner, or other device), the physical size and shape of the patient's teeth may be determined. The deformations of the patterns may be used to determine the shape of the deformed bite block. For example, the patterns provide greater contrast and features on the surface of the bite block as compared to a uniform colored or unpatterned block that aids in creating unique surface features on the bite block that may be identified in 2D images of the block. In some embodiments, the amount of deformation along portions of the pattern may be used to characterize the depth of the bite blocks along those portions, and thus infer local shapes. This may thus allow for the inference of 3D shape from one or more 2D images based on the deformation of the pattern.

In some embodiments, the pattern may be a pattern of non-intersecting lines. In some embodiments, the pattern may be a checkerboard pattern wherein alternating squares of the surface have different colors. In some embodiments, the pattern may be a surface pattern that coats the surface of the bite block. In some embodiments the pattern may have a depth such that the pattern extends 0.1, 0.5, or 1 mm or less into the surface of the bite block. In some embodiments, the pattern may extend through the entire surface of the bite block.

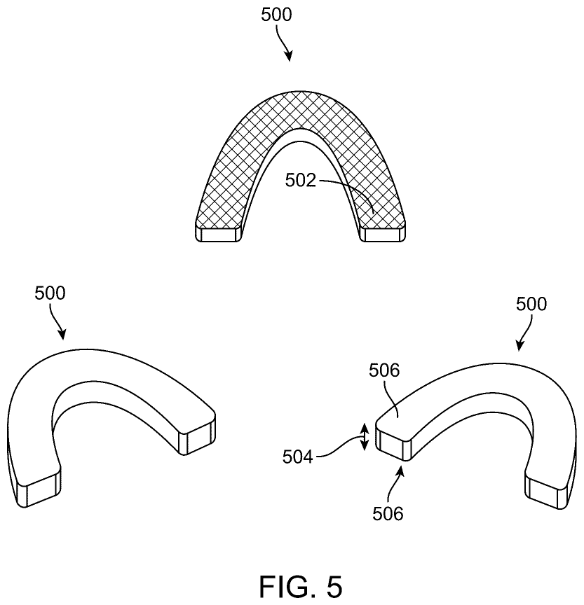

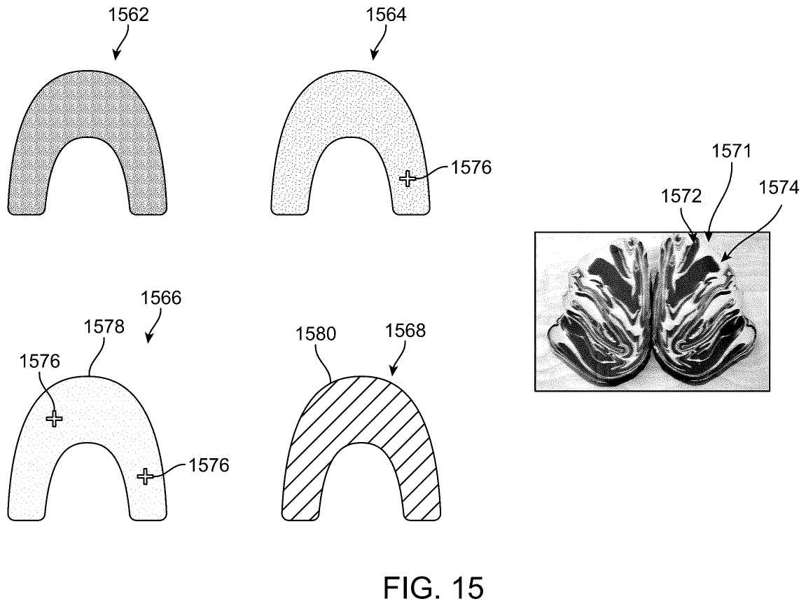

FIG. 15 includes additional depictions of bite blocks of impressioning material with and without various patterns. Bite block 1562 is a uniform bite block without textures. Using such a bite block without a structured light scanning system or other scanning system that projects a pattern on the object to be scanned, such as a bite block, may result in scanning errors as the bite block 1562 may not include sufficient surface features for an effective scan with accurate scan data. Such uniform surfaces, particularly those without a surface texture, are difficult to scan in locations with smooth or uniform surface geometry. For example, the perimeter areas of the bite block impression between the upper and lower impressions, may be difficult to scan because they do not have adequate surface texture, sec for example, in FIG. 6. Such perimeter areas may be smooth or uniform and not include aspects of the tooth impressions. When scanning the impressions, a scanning system may transition between the impression of the upper arch to the impression of the lower arch. During the transition, the scanning system may scan the perimeter areas of the impression that have low surface detail. During the scanning process, when individual frames of scan data are stitched together, the accuracy of low surface detail areas, such as the perimeter, may not be accurately stitched together. The result may be an inaccurate relationship between the impression on one side of the bite block and the other.

In some embodiments, the bite block 1564 may have a random texture. The random texture may be formed by using a mixture of different colored material, such as wax, or an embedded filler, which may include non-wax objects. The embedded filler may be fibers or other particles that are mixed into or otherwise within the bite block material. The distribution of the particles may be homogeneous. In some embodiments, the wax may include particles having a size of between 10 microns and 250 microns, preferably between 10 microns and 150 microns. In some embodiments, the particles may be at least half the minimum scannable feature size of the scanner.

In some embodiments, the particles may be distributed on the surface of the bite block with a density of between 1 and 50 per square millimeter. In some embodiments, the density may be between 1 and 15 per square millimeter. In some embodiments, the particles may be distributed though the volume of the bite block with a density of between 2 and 100 per cubic mm. The use of a relatively low density of particles, as compared to the resolution of a typical intraoral scanner, to capture the impression is achievable, at least in part, because of the process described herein. The bite impression is used to determine the position and orientation of the teeth, in some embodiments, it is used only to determine the position and orientation of the teeth. This may be possible because the actual shapes of the teeth were captured at a relatively high fidelity previously, such as in the initial scan using an intraoral scanner. In the process described herein, the scan of the bite block is used to determine the position and orientation of the teeth at a later time, without a new full intraoral scan. For example, each tooth impression in the bite block scan is matched to a corresponding patient's tooth, such as through surface matching using a surface matching algorithm. Then the position and orientation of the high fidelity tooth model is placed in the position and orientation determined from a matched tooth's occlusal impression.

In some emblements, the particles may be distributed on the surface with a density of at least the minimum scannable feature size of the scanner. In some embodiments, the particles may be distributed on the surface with a density of at least the half the minimum scannable feature size of the scanner.

Bite block 1566 shows an example of sparce spot features. In some embodiments, sparse features may include a mixture of different colored materials, and/or embedded filler, such as non-wax objects. The embedded filler may be particles that are mixed into or otherwise within the material. The distribution of the particles may be homogeneous or non-homogeneous. In some embodiments, the material may include particles having a size of between 100 microns and 1 mm. In some embodiments, the particles may be at least half the minimum scannable feature size of the scanner.

In some embodiments, the particles may be distributed on the surface of the surface with a density of between 10 and 100 per square centimeter. In some embodiments, the particles may be distributed though the volume of the material with a density of between 10 and 1000 per cubic centimeter. In some emblements, the particles may be distributed on the surface with a density that allows between 5 and 50 particles to be observed within a field of view of the scanner during the scanning process.

Bite block 1564 and bite block 1566 also include larger features 1576, which maybe unique features, such as features with a unique shape as compared to the particles or patterns on or in the bite block. The features 1576 may have a size of at least 2 mm in diameter, length, and/or width. The features may be used in combination with any of the other textures discussed herein, such as those depicted in bite blocks 1564, 1566, 1568, 1571, and 500. The features 1576 may be distributed such that between 1 and 6 features are provided or visible on each of the upper and lower surfaces of the bite block.

In some embodiments, the unique features may be added after the impression is taken. For example, unique features may be added to locations on the bite block where teeth impressions are not present. In some embodiments, the unique features may penetrate the bite block such that the unique features extend from an upper surface of the bite block including impressions of the teeth of a first arch toward a lower surface of the block including impressions of the teeth of a second arch (i.e., the unique features may extend partially or entirely through the bite block). In other embodiments, the unique feature may only be on the surface of the bite block. In some embodiments, the material of the bite block may be transparent or partially transparent such that the unique feature or features, while only located on a single side of the bite block, may be observed from both sides. This may allow the scanning system, such as a camera, to observe the position of the unique features from both sides of the bite block.

Bite block 1568 includes a linear texture or linear features 1580. The linear texture or features 1580 may be applied to a surface of the bite block 1568. The linear features may be formed with a coating, such as a wax coating, applied to the bite block, or by apply a coating of any other suitable material. In some embodiments, the linear features may include a pigment applied to the surface of the bite block, such as an edible or otherwise biocompatible pigment. In some embodiments, the linear features may be alternating colors. For example, a first color of linear features may be separated from other linear features of the first color by linear features of a second color or by the underlying color of the bite block. For example, in some embodiments, the linear features may be formed by applying linear features of a single color. In some embodiments, the linear features may be formed by applying alternating colors of linear features. In some embodiments, the linear features may be layers of alternating colors of material. For example, a linear feature may extend through the bite block from a first side to a second side. In some embodiments, the pattern of the feature need not be linear, but may be any other predetermined pattern (e.g., a wave, serpentine, or other curved pattern; a predetermined shape) or a random pattern.

In some embodiments, the width of a linear feature on the surface of the bite block may be measured normal to the sides of the linear feature and may be between 0.25 mm and 5 mm in width, preferable between 1 mm and 3 mm in width. In some embodiments, the width of the linear features may be greater than a minimum feature size of a scanner used to scan the bite block.

Bite block 1571 is an example of a bite block having two different colors, such as a dark color 1572 and a light color 1574 of material mixed together to form a non-homogenous mixture of material, such as wax. The non-homogenous mixture may be formed by placing two different colors of sheets or strips on top of each other and then folding and compressing the sheets three to five times. The sheets and strips may have a thickness of between 1 mm and 3 mm. The sheets may have a ratio of length to width of between 1:1 and 1:4, the sheets may have a ratio of length to width of between 1:4 and 1:10.

In some embodiments, features may be mixed into the bite block during fabrication of the bite block in order to distribute the features throughout the bite block. For example, small spheres having a color different than the color of the bite block material may be mixed in and distributed throughout the bite block.

FIG. 6 shows the bite block 500 after having been impressed upon by the patient's teeth. The bite block 500 includes impressions 604 of the occlusal portions of the patient's teeth. The impressions may include a perimeter 602 that defines the outer edge of the impressions. The impressions may also include the features 606 of the occlusal surfaces of the patient's teeth, such as the cusps, grooves, and ridges of the patient's teeth. In some embodiments, the impression may capture less than 10% of the crown height of the patient's teeth. In some embodiments, the impression may capture between 1 and 3 mm of the crown height starting from the occlusal surface of the patient's teeth.

The impression may be digitized in order to generate a three-dimensional model of the impression. In some embodiments, the bite block impression may be scanned with the 3D scanner such as a structured light 3D scanner. The bite block impression may be sent to a dentist/orthodontist or dental lab for scanning with an intraoral scanner or other three-dimensional scanner.

In some embodiments, 2D photos and/or video of the bite block taken at different angles, captured by the patient, such as through the use of a smartphone, may be used in order to generate a three-dimensional model of the impression. For example, photos of the bite block may be taken from a left, a right, a top, and a bottom location. For example, a two-dimensional photo may be taken from an angle from the right of centerline of the arch, left of the centerline of the arch, from an anterior angle, and a posterior angle. A 2D to 3D conversion process may be used on the photographs in order to generate a three-dimensional model of the impression. Creating a 3D model from multiple 2D photos of an object may use processes such as photogrammetry or SLAM image processing. The first step in this process is to capture a series of overlapping 2D photos of the object from different angles, such as the left, right, anterior, and posterior angles discussed herein. The more photos used, the more accurate the 3D model may be. In these embodiments, the bite block may not need to be sent by the patient to the dentist/orthodontist or dental lab, and the patient can instead simply transmit the photos and/or video to the dentist/orthodontist or dental lab.

The photos are then processed to generate the 3D model. First, common points in the overlapping features in the images are found and used to generate a point cloud. This point cloud is a 3D representation of the object based on the locations of the common points in each image.

A mesh is generated from the point cloud. The mesh is a series of interconnected polygons that represent the surface of the object. In some embodiments, the model of the bite block may be segmented in order to separate the tooth impressions from the non-tooth impression portions of the bite block. In some embodiments, the individual teeth impressions within the bite block may be segmented from each other. Segmenting may be performed manually or automatically. Manual segmentation involves tracing the outline of the teeth impressions using a digital pen or mouse, while automatic segmentation uses algorithms to identify and separate the teeth impressions from the surrounding structures.

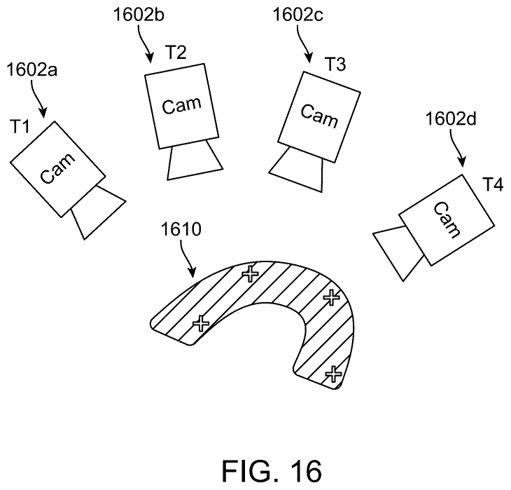

FIG. 16 shows an example of a scanning process that use images from a 2D camera or cameras 1602 to generate a 3D model. The process may include imaging with camera 1602 at several camera locations. The scanning process may begin with capturing images of the bite block impression 1610 using one or more 2D cameras which may be mounted on a moving platform, and the platform may be rotated manually or automatically (e.g., using a motor, a robotic arm). In some embodiments, the camera or cameras may be still while the bite block impression moves relative to the cameras (e.g., by having the bite block impression be on a moving platform). A first image may be captured at camera location 1602a, a second image may be captured at camera location 1602b, a third image may be captured at camera location 1602c, and so on. In some embodiments, as few as two images may be captured and as many as 1000 images may be captured from multiple locations and/or camera orientations for each side of the bite block impression 1610.

Key features, such as points, edges, or textures, such as the features and/or textures discussed above are identified within the images. Then corresponding features between consecutive or non-consecutive images are matched or associated with each other. The pose of the camera, such as the camera position and/or orientation relative to the bite block, for each frame may be estimated using the matched features. A depth map may be generated based on the detected features. With each additional image, the depth map may be updated to refine features, such as the surface geometry of the impressions, and/or to add additional features not included in the map. Using the accumulated 2D images and the poses of the camera, a 3D model of the surface of the bite block impression 1610 may be generated. In some embodiments, photogrammetry or SLAM imaging may be used to generate a 3D model of the bite block.

The process discussed above may be repeated on a second side of the bite block impression, for example, the bite block impression may be flipped over. The models of the two sides may be registered together based on, for example, the location of one or more unique features that may be visible from both sides of the bite block.

In some embodiments, rather than create a second model for the second side of the impression, the process may continue after the impression is flipped over such that the 3D model of the impression is refined and added to as additional 2D images are captured until the upper and lower impressions are captured.

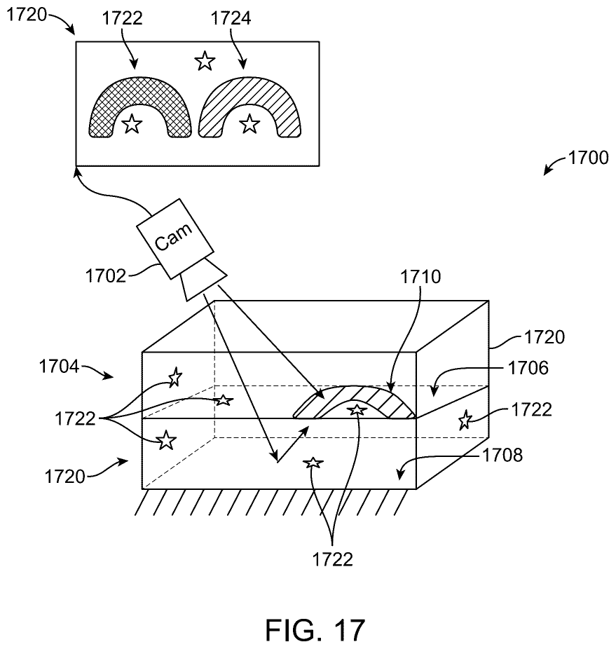

In some embodiments, the upper and lower impressions of a impression may be scanned simultaneously. For example, as depicted in FIG. 17. An apparatus in including a support, such as a transparent surface, such as glass, or a net may support a impression 1710 above or in front of a mirrored surface 1708. In some embodiments, the support may be a post or other structure that supports the impression above or in front of the mirror. A camera 1702 may then capture images of the impression 1710. Each image may include a view of at least a portion of the upper surface of the impression, which may include an impression of a first arch of the patient, and of at least a portion the lower surface of the impression, which may include an impression of a second arch of the patient. The camera 1702 and the impression 1710 may move relative to each other. For example, one may remain stationary with the other moves. The camera may capture images of the impression from multiple positions and/or orientations and the images may be used to build a 3D surface model of the impression using the process discussed above with respect to FIG. 16 or other processes.

In some embodiments, the apparatus 1704 may include sidewalls 1720 that may surround the support 1706 and the mirror 1708. In some embodiments, one or more fiducials 1722 may be located on the mirror, surface, or sidewalls that may be captured by the camera to aid in determining the location of the camera relative to the apparatus 1704 and/or the impression 1710. For example, the fiducials may be in known locations.

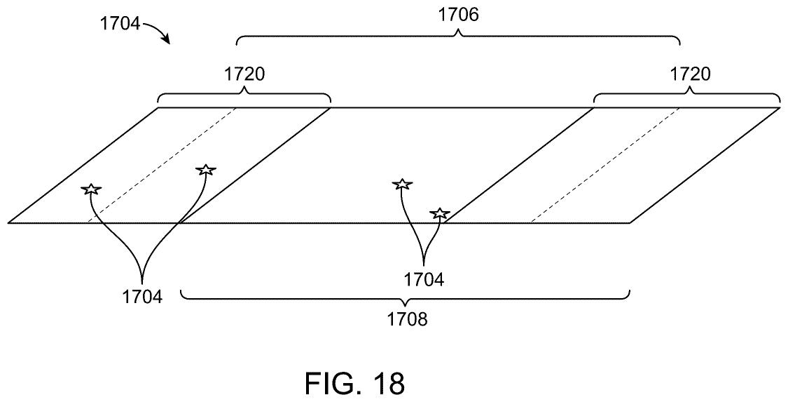

In some embodiments, the apparatus may be configurable between an expanded configuration for scanning, such as depicted in FIG. 17 and a collapsed configuration, such as depicted in FIG. 18, wherein sidewalls 1720 are folded flat with the mirror 1708 and the support 1706.

After the 3D model of the bite block impressions is generated, the tooth models of the initial scan of the patient's dentition may be used along with the bite block model in order to generate a 3D model of the current position of the patient's teeth. Teeth are generally rigid structures whose overall shapes do not change very much over time. While in orthodontic treatment the positions of the teeth may move, the shapes of the teeth may not change significantly. Because the shapes of the teeth do not change, the 3D teeth of the patient's initial scan may be used and aligned with the shapes of the impressions of the patient's teeth from the bite block in order to align the patient's teeth in their current position based on the bite block impressions.

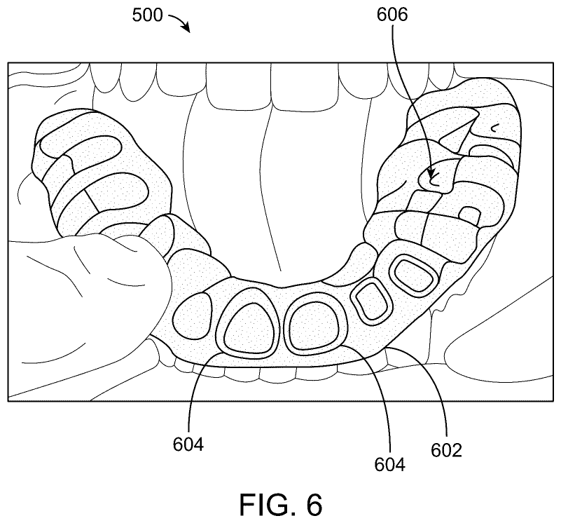

FIG. 7 depicts views of a three-dimensional model 700 of the patient's dentition based on a scan of the patient's teeth. This model may have been previously segmented such that each individual tooth is separated from every other tooth. A segmented model may also include the location and orientation of the patient's teeth in 6 degrees of freedom, which may be modified. The 6 degrees of freedom may include three orthogonal position locations that locate the position of the tooth in three-dimensional space and three orthogonal rotational angles that determine the orientation of the tooth in three-dimensional space.

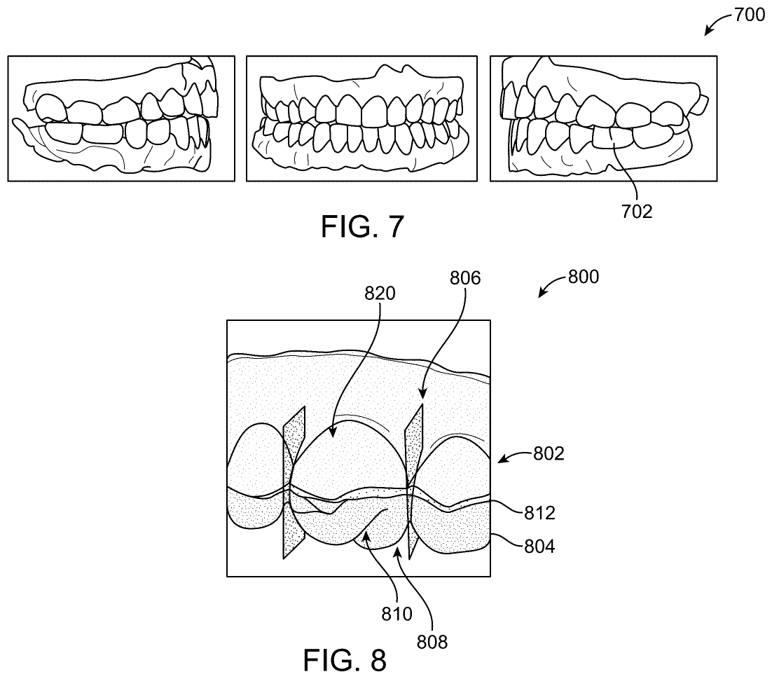

FIG. 8 depicts an alignment of the initial 3D model of the patient's teeth 802 with the model of the impression 804. One or more segmentation planes 806 may be generated and/or visualized to depict the separation or segmentation boundaries between adjacent teeth of the patient's dentition from the initial scan 802. For example, with respect to tooth 820, the shape of the occlusal portion of the patient's teeth including the cusps 808, grooves 810, and other surface features of the patient's tooth 820 may be matched to a corresponding surface shape on the bite block impression model 804 in order to determine the position and orientation of the patient's tooth 820 at the time the impression was made. In some embodiments, the outline of the impression 812 may be used in order to find a corresponding outline on a patient's tooth in order to locate the position of the patient's tooth at the time the impression was made.

After the location and orientation of each tooth of the patient's dentition is determined based on the impression 804, a three-dimensional model of the patient's teeth may be generated.

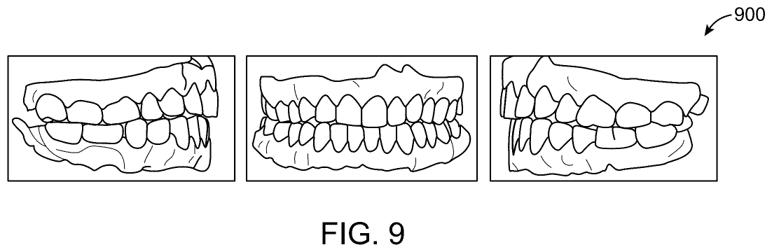

FIG. 9 depicts views of a three-dimensional model 900 of the patient's dentition based on the initial teeth from the initial scan after locating and orientating them according to the tooth locations in the digital model of the bite block impressions. The updated 3D model may be used to generate a new treatment plan or retainers.

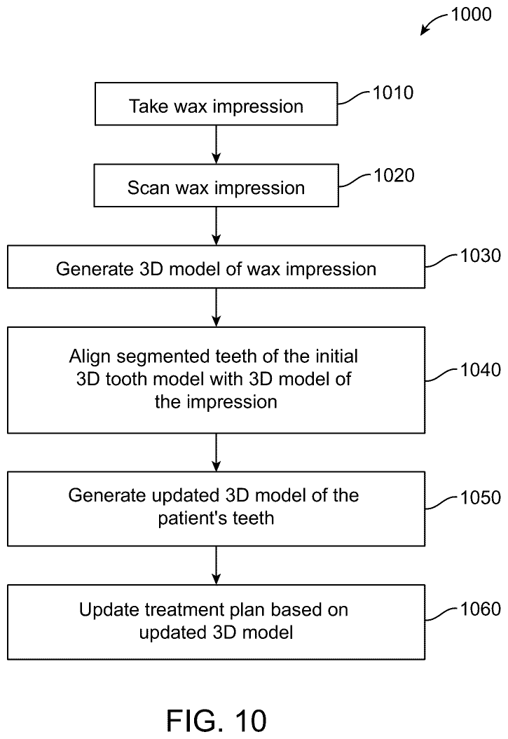

FIG. 10 depicts a method 1000 of using bite block impressions for remote progress tracking and updating treatment plans when treatment is off track. The remote progress tracking may begin with an indication that the patient's orthodontic treatment is off track, such as an aligner not fitting properly or may be part of a regular progress tracking process wherein the positions of the patient's teeth are checked regularly during treatment. In some embodiments, alternative methods of determining tooth position (which may be less accurate) may be used to initially determine that a patient's teeth may be off track, such as through 2D imaging of the patient's teeth. In some embodiments, a patient may regularly take 2D images of their teeth. Current tooth positions may be determined based on the 2D images and those current tooth positions may be checked against expected tooth positions for the current stage of the treatment plan. If they do not appear to match, then bite blocks may be sent to the patient for acquiring an impression.

At block 1010, the patient may take a bite block impression of their teeth. Such as by using any of the bite blocks discussed herein, such as those shown and described with respect to FIGS. 5 and 15. The resulting bite block impression captures the shape and position of the occlusal surfaces of the patient's teeth and bite. The shape and position of the occlusal surfaces may be used to generate a full 3D model of the patient's dentition in the teeth's current arrangement based on the previously scanned 3D model, as discussed herein.

The thickness of the bite block may be based on an expected depth to which the occlusal portions of the teeth intrude into the bite block. In some embodiments, the patient's teeth may protrude only 1 mm or less into the bite block in order to generate an acceptable depth to capture the occlusal features of the patient's teeth in a scan. In some embodiments, the patient's teeth may protrude 2 mm or less into the bite block. In some embodiments, only the occlusal surface of the teeth penetrate into the surface of the bite block. In some embodiments, less than 10% of the crown height of the patient's tooth penetrates the surface of the bite block. In some embodiments, the teeth press into the bite block a depth sufficient to generate an impression of the cusps and grooves of the patient's teeth. In some embodiments, less than 10% of the surface area of the patient's tooth is compressed into the bite block.

The bite block may have a pattern on one or both occlusal surfaces of the bite block. The pattern may be a grid pattern, such as the grid pattern depicted as pattern 502 in FIG. 5. In some embodiments, the pattern may be a pattern of dots. The pattern may help in digitizing and scanning the impression.

In some embodiments, the pattern may be a pattern of non-intersecting lines. In some embodiments, the pattern may be a checkerboard pattern wherein alternating squares of the surface have different colors. In some embodiments, the pattern may be a surface pattern that coats the surface of the bite block. In some embodiments the pattern may have a depth such that the pattern extends 0.1, 0.5, or 1 mm or less into the surface of the bite block. In some embodiments, the pattern may extend through the entire surface of the bite block.

In some embodiments, features may be mixed into the bite block during fabrication of the bite block in order to distribute the features throughout the bite block. For example, small spheres having a color different than the color of the may be mixed in and distributed throughout the bite block.

At block 1020 the bite block impression is scanned. The impression may be digitized or scanned in order to generate a three-dimensional model of the impression. In some embodiments, the bite block impression may be scanned with the 3D scanner such as a structured light 3D scanner. The bite block impression may be sent to a dentist or dental lab for scanning with an intraoral scanner or other three-dimensional scanner. In some embodiments, 2D photos of the bite block taken at different angles, captured by the patient, such as through the use of a smartphone, may be used in order to generate a three-dimensional model of the impression. For example, photos of the bite block may be taken from a left, the right, a top, and a bottom location. For example, using the directions of the mouth a two-dimensional photo may be taken from an angle from the right of centerline of the arch, left of the centerline of the arch, from an anterior angle and a posterior angle. A 2D to 3D conversion process may be used on the photographs in order to generate a three-dimensional model of the impression. Creating a 3D model from multiple 2D photos of an object may use processes such as photogrammetry or SLAM image processing. The first step in this process is to capture a series of overlapping 2D photos of the object from different angles, such as the left, right, anterior, and posterior angles discussed herein. The more photos used, the more accurate the 3D model may be.

In some embodiments, the bite block is used to determine the positions of the patient's teeth in each arch, but not the true bite which may include arch to arch registration, such as used to model articulation.

At block 1030 a 3D model of the patient's dentition is generated from the scan data. In some embodiments, the scanning and generating of the 3D model may take place iteratively. In some embodiments, the scanning and generating of the 3D model may take place simultaneously or in parallel. The photos are then processed to generate the 3D model. First, common points in the overlapping features in the images are found and used to generate a point cloud. This point cloud is a 3D representation of the object based on the locations of the common points in each image.

A mesh is generated from the point cloud. The mesh is a series of interconnected polygons that represent the surface of the object. In some embodiments, the model of the bite block may be segmented in order to separate the tooth impressions from the non-tooth impression portions of the bite block. In some embodiments, the individual teeth impressions within the bite block may be segmented from each other. Segmenting may be performed manually or automatically. Manual segmentation involves tracing the outline of the teeth impressions using a digital pen or mouse, while automatic segmentation uses algorithms to identify and separate the teeth impressions from the surrounding structures.

At block 1040, the segmented teeth of an initial scan of the patient's dentition such as a scan generated before the beginning of orthodontic treatment or a scan generated at an earlier time during treatment may be aligned with the 3D model of the bite block impression. After the 3D model of the bite block impressions is generated, the tooth models of the initial scan of the patient's dentition may be used along with the bite block model in order to generate a 3D model of the current position of the patient's teeth. Teeth are generally rigid structures whose overall shapes do not change very much over time. While in orthodontic treatment the positions of the teeth may move, the shapes of the teeth may not change significantly. Because the shapes of the teeth do not change, the 3D teeth of the patient's initial scan may be used and aligned with the shapes of the impressions of the patient's teeth from the bite block in order to align the patient's teeth in their current position based on the bite block impressions.

At block 1050, an updated 3D model of the patient's dentition is generated based on the positions and orientations the patient's teeth during the bite imprisoning. The three-dimensional model of the patient's dentition is based on the initial teeth from the initial scan after locating and orientating them according to the tooth locations in the digital model of the bite block impressions.

In some embodiments, such as when the patient's treatment is on track, such as when the teeth are within an acceptable deviation from their planned position at the stage of treatment, the process may end at block 1050. If the teeth are not where they are expected to be at the stage of treatment, such as deviating greater than a threshold deviation of translation and/or rotational displacement, then the process may proceed to block 1060.

At block 1060 an updated treatment plan may be generated. The updated 3D model may be used to generate a new treatment plan. The new treatment plan may use the updated, current positions of the teeth of the patient's dentition and the initial target position to generate an updated treatment plan to move the patient's teeth form the new initial position toward the initial target position or an updated target position, as described herein, such as with respect to FIGS. 1-5. In some embodiments, the updated target position may be different from the initial target position (e.g., determined based on how the treatment is progressing for the patient). In some embodiments, such as when the teeth are lagging from the expected position, an updated treatment plan may include the patient wearing an aligner for one or more stages of treatment for a longer period of time than the initial treatment plan called for. In some embodiments, an update treatment plan may include removing one or more stages from the initial treatment plan, such as by instructing the patient to skip one or more aligner stages. In some embodiments, a revised or updated treatment plan may include restaging of the teeth and/or sending new or additional aligners for new or additional stages of treatment. In some embodiments, the revised treatment plan may include one or more additional stages of treatment, and corresponding aligners, to bring the teeth back on track with the original treatment plan, at which point the patient may continue treatment based on the initial treatment plan and corresponding aligners provided with the original treatment plan. In some embodiments, the teeth of the patient's dentition may be in a state such that the system disclosed herein sends a recommendation to the patient to visit their treating professional, such as their dentist of orthodontist, for evaluation, rescan with an intraoral scanner, etc.

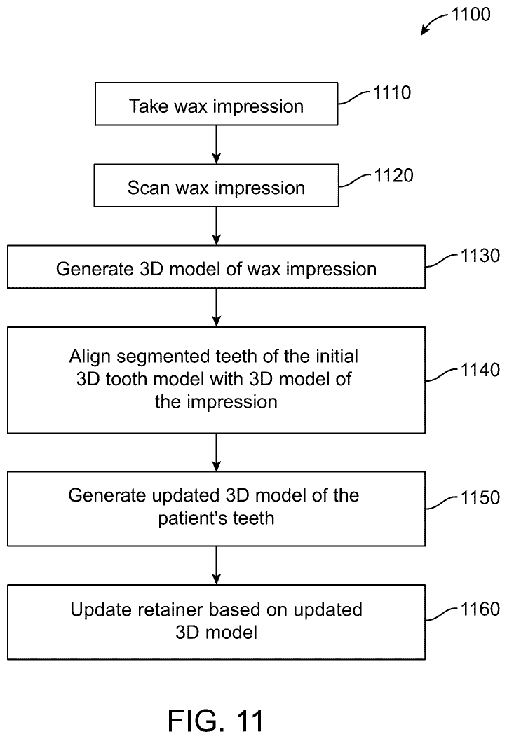

FIG. 11 depicts a method 1100 of using bite block impressions for remote post treatment tooth movement tracking. Remote post treatment tooth movement tracking may begin with an indication that the patient's teeth have moved after completion of orthodontic treatment, such as when a retainer is not fitting properly or may be part of a regular remote post treatment tooth movement tracking process wherein the position of the patient's teeth are checked regularly after treatment. In some embodiments, less accurate methods of determining tooth position may be used to initially determine that a patient's teeth may have shifted, such as through 2D imaging of the patient's teeth. In some embodiments, a patient may regularly take 2D images of their teeth. Tooth positions may be determined based on the 2D images and those current positions may be checked against the expected post treatment position. If they do not appear to match, then bite blocks may be sent to the patient for imprisoning.

At block 1110, the patient may take a bite block impression of their teeth. The process may be similar or the same as described with respect to block 1010 of FIG. 10.

At block 1120 the bite block impression is scanned. The impression may be digitized or scanned in order to generate a three-dimensional model of the impression. In some embodiments, the bite block impression may be scanned with the 3D scanner such as a structured light 3D scanner. The bite block impression may be sent to a dentist or dental lab for scanning with an intraoral scanner or other three-dimensional scanner. In some embodiments, two-dimensional photos of the bite block taken at different angles, captured by the patient, such as through the use of a smartphone, may be used in order to generate a three-dimensional model of the impression. For example, photos of the bite block maybe taken from a left, the right, a top, and a bottom location. For example, using the directions of the mouth a two-dimensional photo may be taken from an angle from the right of centerline of the arch, left of the centerline of the arch, from an anterior angle and a posterior angle. A 2D to 3D conversion process may be used on the photographs in order to generate a three-dimensional model of the impression. Creating a 3D model from multiple 2D photos of an object may use processes such as photogrammetry or SLAM image processing. The first step in this process is to capture a series of overlapping 2D photos of the object from different angles, such as the left, right, anterior, and posterior angles discussed herein. The more photos used, the more accurate the 3D model may be.

In some embodiments, the bite block is used to determine the positions of the patient's teeth in each arch, but not the true bite which may include arch to arch registration, such as used to model articulation.

At block 1130 a 3D model of the patient's dentition is generated from the scan data. In some embodiments, the scanning and generating of the 3D model may take place iteratively. In some embodiments, the scanning and generating of the 3D model may take place simultaneously or in parallel. The photos are then processed to generate the 3D model. First, common points in the overlapping features in the images are found and used to generate a point cloud. This point cloud is a 3D representation of the object based on the locations of the common points in each image.

A mesh is generated from the point cloud. The mesh is a series of interconnected polygons that represent the surface of the object. In some embodiments, the model of the bite block may be segmented in order to separate the tooth impressions from the non-tooth impression portions of the bite block. In some embodiments, the individual teeth impressions within the bite block may be segmented from each other. Segmenting may be performed manually or automatically. Manual segmentation involves tracing the outline of the teeth impressions using a digital pen or mouse, while automatic segmentation uses algorithms to identify and separate the teeth impressions from the surrounding structures.

At block 1140, the segmented teeth of an initial scan of the patient's dentition such as a scan generated before the beginning of orthodontic treatment or a scan generated at an earlier time during treatment may be aligned with the 3D model of the bite block impression. After the 3D model of the bite block impressions is generated, the tooth models of the initial scan of the patient's dentition may be used along with the bite block model in order to generate a 3D model of the current position of the patient's teeth. Teeth are generally rigid structures whose overall shapes do not change very much over time. While in orthodontic treatment the positions of the teeth may move, the shapes of the teeth may not change significantly. Because the shapes of the teeth do not change, the 3D teeth of the patient's initial scan may be used and aligned with the shapes of the impressions of the patient's teeth from the bite block in order to align the patient's teeth in their current position based on the bite block impressions.

At block 1150, an updated 3D model of the patient's dentition is generated based on the positions and orientations the patient's teeth during the bite imprisoning. The three-dimensional model of the patient's dentition is based on the initial teeth from the initial scan after locating and orientating them according to the tooth locations in the digital model of the bite block impressions.

At block 1160 an updated retainer be generated. The updated 3D model may be used to generate a retainer to hold the patient's teeth in the new position. In some embodiments, a new treatment plan may be generated. The new treatment plan may use the updated positions as the initial position and a treatment plan may be generated to move the patient's teeth form the new initial position toward the target position or an updated target position, as described herein, such as with respect to FIGS. 1-5.

### Computing System

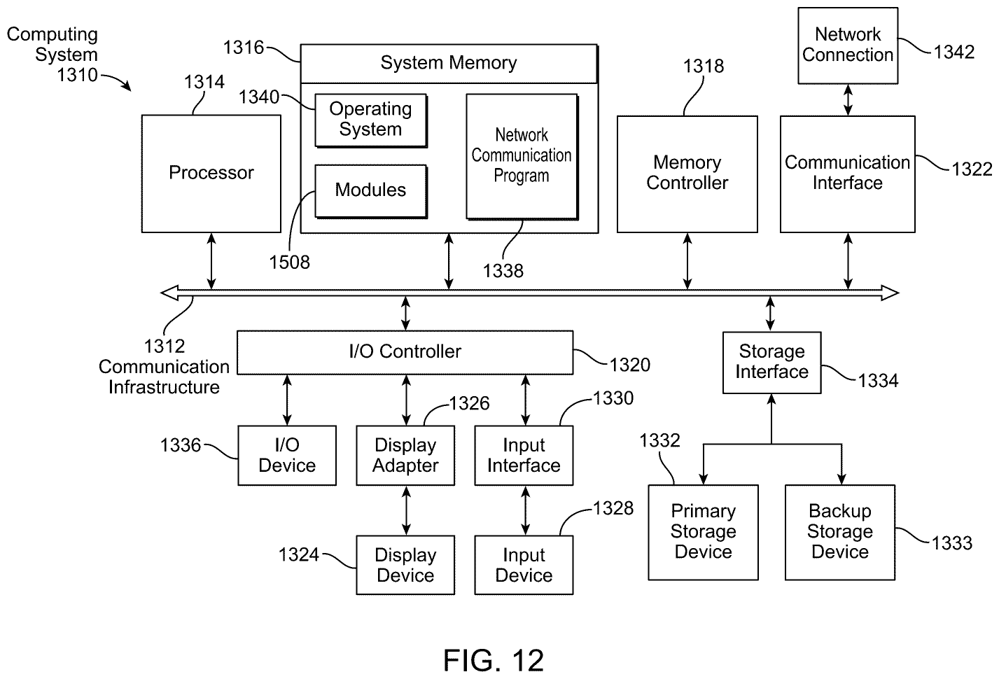

FIG. 12 is a block diagram of an example computing system 1310 capable of implementing one or more of the embodiments described and/or illustrated herein. For example, all or a portion of computing system 1310 may perform and/or be a means for performing, either alone or in combination with other elements, one or more of the steps described herein (such as one or more of the steps illustrated in FIGS. 1-10. All or a portion of computing system 1310 may also perform and/or be a means for performing any other steps, methods, or processes described and/or illustrated herein.

Computing system 1310 broadly represents any single or multi-processor computing device or system capable of executing computer-readable instructions. Examples of computing system 1310 include, without limitation, workstations, laptops, client-side terminals, servers, distributed computing systems, handheld devices, or any other computing system or device. In its most basic configuration, computing system 1310 may include at least one processor 1314 and a system memory 1316.

Processor 1314 generally represents any type or form of physical processing unit (e.g., a hardware-implemented central processing unit) capable of processing data or interpreting and executing instructions. In certain embodiments, processor 1314 may receive instructions from a software application or module. These instructions may cause processor 1314 to perform the functions of one or more of the example embodiments described and/or illustrated herein.

System memory 1316 generally represents any type or form of volatile or non-volatile storage device or medium capable of storing data and/or other computer-readable instructions. Examples of system memory 1316 include, without limitation, Random Access Memory (RAM), Read Only Memory (ROM), flash memory, or any other suitable memory device. Although not required, in certain embodiments computing system 1310 may include both a volatile memory unit (such as, for example, system memory 1316) and a non-volatile storage device (such as, for example, primary storage device 1332, as described in detail below).

In some examples, system memory 1316 may store and/or load an operating system 1340 for execution by processor 1314. In one example, operating system 1340 may include and/or represent software that manages computer hardware and software resources and/or provides common services to computer programs and/or applications on computing system 1310. Examples of operating system 1340 include, without limitation, LINUX, JUNOS, MICROSOFT WINDOWS, WINDOWS MOBILE, MAC OS, APPLE'S IOS, UNIX, GOOGLE CHROME OS, GOOGLE'S ANDROID, SOLARIS, variations of one or more of the same, and/or any other suitable operating system.

In certain embodiments, example computing system 1310 may also include one or more components or elements in addition to processor 1314 and system memory 1316. For example, as illustrated in FIG. 12, computing system 1310 may include a memory controller 1318, an Input/Output (I/O) controller 1320, and a communication interface 1322, each of which may be interconnected via a communication infrastructure 1312. Communication infrastructure 1312 generally represents any type or form of infrastructure capable of facilitating communication between one or more components of a computing device. Examples of communication infrastructure 1312 include, without limitation, a communication bus (such as an Industry Standard Architecture (ISA), Peripheral Component Interconnect (PCI), PCI Express (PCIe), or similar bus) and a network.

Memory controller 1318 generally represents any type or form of device capable of handling memory or data or controlling communication between one or more components of computing system 1310. For example, in certain embodiments memory controller 1318 may control communication between processor 1314, system memory 1316, and I/O controller 1320 via communication infrastructure 1312.

I/O controller 1320 generally represents any type or form of module capable of coordinating and/or controlling the input and output functions of a computing device. For example, in certain embodiments I/O controller 1320 may control or facilitate transfer of data between one or more elements of computing system 1310, such as processor 1314, system memory 1316, communication interface 1322, display adapter 1326, input interface 1330, and storage interface 1334.

As illustrated in FIG. 12, computing system 1310 may also include at least one display device 1324 coupled to I/O controller 1320 via a display adapter 1326. Display device 1324 generally represents any type or form of device capable of visually displaying information forwarded by display adapter 1326. Similarly, display adapter 1326 generally represents any type or form of device configured to forward graphics, text, and other data from communication infrastructure 1312 (or from a frame buffer, as known in the art) for display on display device 1324.

As illustrated in FIG. 12, example computing system 1310 may also include at least one input device 1328 coupled to I/O controller 1320 via an input interface 1330. Input device 1328 generally represents any type or form of input device capable of providing input, either computer or human generated, to example computing system 1310. Examples of input device 1328 include, without limitation, a keyboard, a pointing device, a speech recognition device, variations or combinations of one or more of the same, and/or any other input device.

Additionally or alternatively, example computing system 1310 may include additional I/O devices. For example, example computing system 1310 may include I/O device 1336. In this example, I/O device 1336 may include and/or represent a user interface that facilitates human interaction with computing system 1310. Examples of I/O device 1336 include, without limitation, a computer mouse, a keyboard, a monitor, a printer, a modem, a camera, a scanner, a microphone, a touchscreen device, variations or combinations of one or more of the same, and/or any other I/O device.

Communication interface 1322 broadly represents any type or form of communication device or adapter capable of facilitating communication between example computing system 1310 and one or more additional devices. For example, in certain embodiments communication interface 1322 may facilitate communication between computing system 1310 and a private or public network including additional computing systems. Examples of communication interface 1322 include, without limitation, a wired network interface (such as a network interface card), a wireless network interface (such as a wireless network interface card), a modem, and any other suitable interface. In at least one embodiment, communication interface 1322 may provide a direct connection to a remote server via a direct link to a network, such as the Internet. Communication interface 1322 may also indirectly provide such a connection through, for example, a local area network (such as an Ethernet network), a personal area network, a telephone or cable network, a cellular telephone connection, a satellite data connection, or any other suitable connection.

In certain embodiments, communication interface 1322 may also represent a host adapter configured to facilitate communication between computing system 1310 and one or more additional network or storage devices via an external bus or communications channel. Examples of host adapters include, without limitation, Small Computer System Interface (SCSI) host adapters, Universal Serial Bus (USB) host adapters, Institute of Electrical and Electronics Engineers (IEEE) 1394 host adapters, Advanced Technology Attachment (ATA), Parallel ATA (PATA), Serial ATA (SATA), and External SATA (eSATA) host adapters, Fibre Channel interface adapters, Ethernet adapters, or the like. Communication interface 1322 may also allow computing system 1310 to engage in distributed or remote computing. For example, communication interface 1322 may receive instructions from a remote device or send instructions to a remote device for execution.

In some examples, system memory 1316 may store and/or load a network communication program 1338 for execution by processor 1314. In one example, network communication program 1338 may include and/or represent software that enables computing system 1310 to establish a network connection 1342 with another computing system (not illustrated in FIG. 12) and/or communicate with the other computing system by way of communication interface 1322. In this example, network communication program 1338 may direct the flow of outgoing traffic that is sent to the other computing system via network connection 1342. Additionally or alternatively, network communication program 1338 may direct the processing of incoming traffic that is received from the other computing system via network connection 1342 in connection with processor 1314.

Although not illustrated in this way in FIG. 12, network communication program 1338 may alternatively be stored and/or loaded in communication interface 1322. For example, network communication program 1338 may include and/or represent at least a portion of software and/or firmware that is executed by a processor and/or Application Specific Integrated Circuit (ASIC) incorporated in communication interface 1322.

As illustrated in FIG. 12, example computing system 1310 may also include a primary storage device 1332 and a backup storage device 1333 coupled to communication infrastructure 1312 via a storage interface 1334. Storage devices 1332 and 1333 generally represent any type or form of storage device or medium capable of storing data and/or other computer-readable instructions. For example, storage devices 1332 and 1333 may be a magnetic disk drive (e.g., a so-called hard drive), a solid state drive, a floppy disk drive, a magnetic tape drive, an optical disk drive, a flash drive, or the like. Storage interface 1334 generally represents any type or form of interface or device for transferring data between storage devices 1332 and 1333 and other components of computing system 1310.

In certain embodiments, storage devices 1332 and 1333 may be configured to read from and/or write to a removable storage unit configured to store computer software, data, or other computer-readable information. Examples of suitable removable storage units include, without limitation, a floppy disk, a magnetic tape, an optical disk, a flash memory device, or the like. Storage devices 1332 and 1333 may also include other similar structures or devices for allowing computer software, data, or other computer-readable instructions to be loaded into computing system 1310. For example, storage devices 1332 and 1333 may be configured to read and write software, data, or other computer-readable information. Storage devices 1332 and 1333 may also be a part of computing system 1310 or may be a separate device accessed through other interface systems.

Many other devices or subsystems may be connected to computing system 1310. Conversely, all of the components and devices illustrated in FIG. 12 need not be present to practice the embodiments described and/or illustrated herein. The devices and subsystems referenced above may also be interconnected in different ways from that shown in FIG. 12. Computing system 1310 may also employ any number of software, firmware, and/or hardware configurations. For example, one or more of the example embodiments disclosed herein may be encoded as a computer program (also referred to as computer software, software applications, computer-readable instructions, or computer control logic) on a computer-readable medium.

The term “computer-readable medium,” as used herein, generally refers to any form of device, carrier, or medium capable of storing or carrying computer-readable instructions. Examples of computer-readable media include, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

The computer-readable medium containing the computer program may be loaded into computing system 1310. All or a portion of the computer program stored on the computer-readable medium may then be stored in system memory 1316 and/or various portions of storage devices 1332 and 1333. When executed by processor 1314, a computer program loaded into computing system 1310 may cause processor 1314 to perform and/or be a means for performing the functions of one or more of the example embodiments described and/or illustrated herein. Additionally or alternatively, one or more of the example embodiments described and/or illustrated herein may be implemented in firmware and/or hardware. For example, computing system 1310 may be configured as an Application Specific Integrated Circuit (ASIC) adapted to implement one or more of the example embodiments disclosed herein.

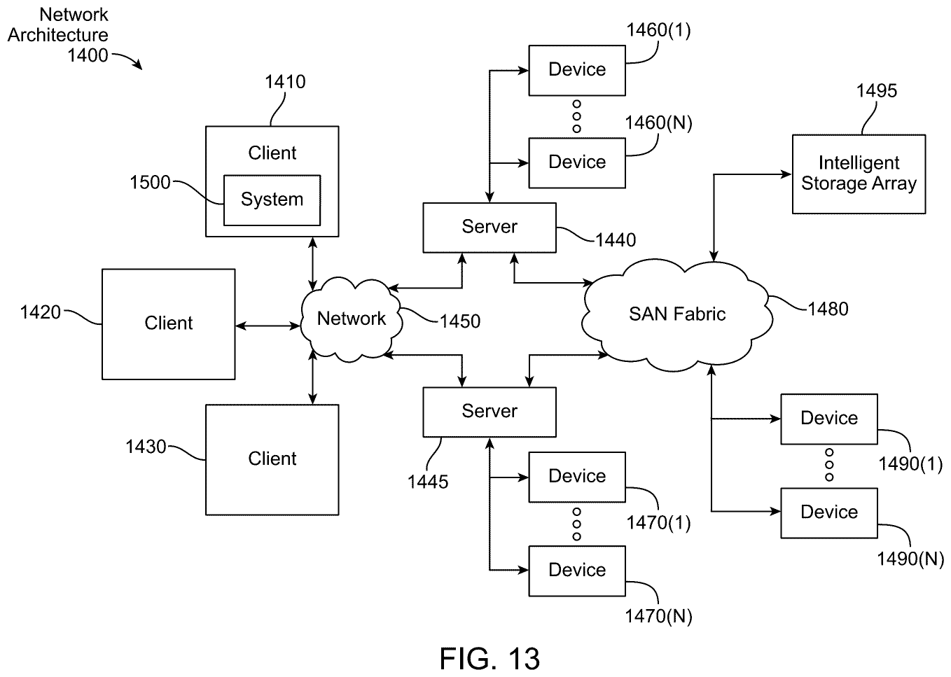

FIG. 13 is a block diagram of an example network architecture 1400 in which client systems 1410, 1420, and 1430 and servers 1440 and 1445 may be coupled to a network 1450. As detailed above, all or a portion of network architecture 1400 may perform and/or be a means for performing, cither alone or in combination with other elements, one or more of the steps disclosed herein (such as one or more of the steps illustrated in FIG. 4-11). All or a portion of network architecture 1400 may also be used to perform and/or be a means for performing other steps and features set forth in the instant disclosure.

Client systems 1410, 1420, and 1430 generally represent any type or form of computing device or system, such as example computing system 1310 in FIG. 12. Similarly, servers 1440 and 1445 generally represent computing devices or systems, such as application servers or database servers, configured to provide various database services and/or run certain software applications. Network 1450 generally represents any telecommunication or computer network including, for example, an intranet, a WAN, a LAN, a PAN, or the Internet. In one example, client systems 1410, 1420, and/or 1430 and/or servers 1440 and/or 1445 may include all or a portion of system 500 from FIG. 5.

As illustrated in FIG. 13, one or more storage devices 1460(1)-(N) may be directly attached to server 1440. Similarly, one or more storage devices 1470(1)-(N) may be directly attached to server 1445. Storage devices 1460(1)-(N) and storage devices 1470(1)-(N) generally represent any type or form of storage device or medium capable of storing data and/or other computer-readable instructions. In certain embodiments, storage devices 1460(1)-(N) and storage devices 1470(1)-(N) may represent Network-Attached Storage (NAS) devices configured to communicate with servers 1440 and 1445 using various protocols, such as Network File System (NFS), Server Message Block (SMB), or Common Internet File System (CIFS).

Servers 1440 and 1445 may also be connected to a Storage Area Network (SAN) fabric 1480. SAN fabric 1480 generally represents any type or form of computer network or architecture capable of facilitating communication between a plurality of storage devices. SAN fabric 1480 may facilitate communication between servers 1440 and 1445 and a plurality of storage devices 1490(1)-(N) and/or an intelligent storage array 1495. SAN fabric 1480 may also facilitate, via network 1450 and servers 1440 and 1445, communication between client systems 1410, 1420, and 1430 and storage devices 1490(1)-(N) and/or intelligent storage array 1495 in such a manner that devices 1490(1)-(N) and array 1495 appear as locally attached devices to client systems 1410, 1420, and 1430. As with storage devices 1460(1)-(N) and storage devices 1470(1)-(N), storage devices 1490(1)-(N) and intelligent storage array 1495 generally represent any type or form of storage device or medium capable of storing data and/or other computer-readable instructions.

In certain embodiments, and with reference to example computing system 1310 of FIG. 12, a communication interface, such as communication interface 1322 in FIG. 12, may be used to provide connectivity between each client system 1410, 1420, and 1430 and network 1450. Client systems 1410, 1420, and 1430 may be able to access information on server 1440 or 1445 using, for example, a web browser or other client software. Such software may allow client systems 1410, 1420, and 1430 to access data hosted by server 1440, server 1445, storage devices 1460(1)-(N), storage devices 1470(1)-(N), storage devices 1490(1)-(N), or intelligent storage array 1495. Although FIG. 12 depicts the use of a network (such as the Internet) for exchanging data, the embodiments described and/or illustrated herein are not limited to the Internet or any particular network-based environment.

In at least one embodiment, all or a portion of one or more of the example embodiments disclosed herein may be encoded as a computer program and loaded onto and executed by server 1440, server 1445, storage devices 1460(1)-(N), storage devices 1470(1)-(N), storage devices 1490(1)-(N), intelligent storage array 1495, or any combination thereof. All or a portion of one or more of the example embodiments disclosed herein may also be encoded as a computer program, stored in server 1440, run by server 1445, and distributed to client systems 1410, 1420, and 1430 over network 1450.

As detailed above, computing system 1310 and/or one or more components of network architecture 1400 may perform and/or be a means for performing, either alone or in combination with other elements, one or more steps of an example method for virtual care.

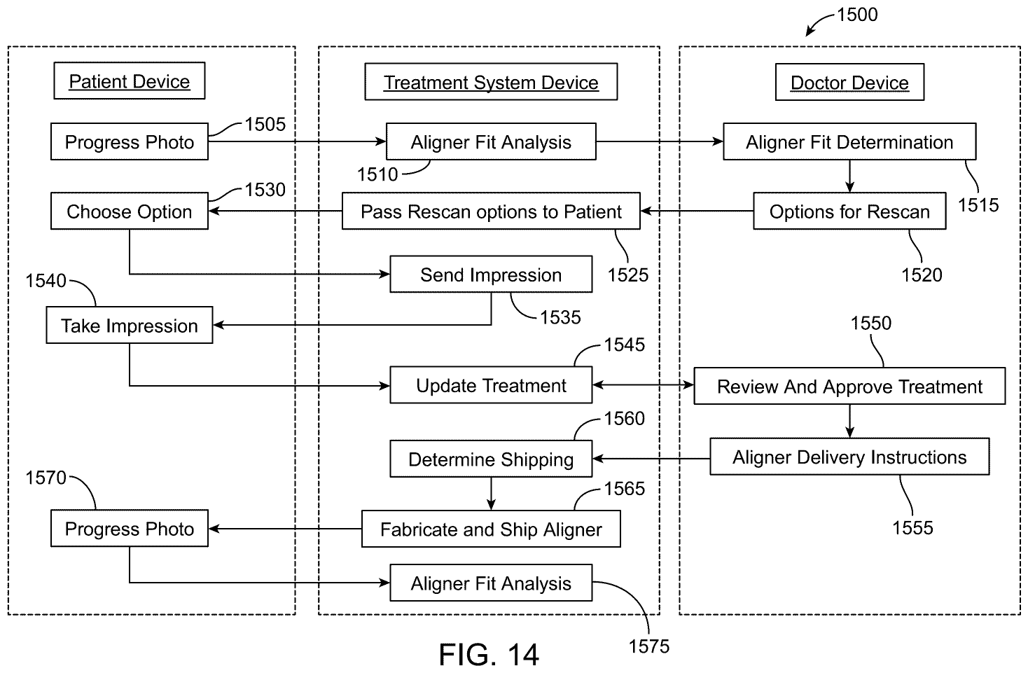

FIG. 14 depicts a process 1500 for using impressions to update a treatment plan. The method 1500 may start a block 1505, wherein a patient takes a progress photo. A progress photo may be a 2D image of the patient wearing an aligner. The photo may be used to determine whether or how well the aligner is fitting on the patient's teeth. In some embodiments, at block 1505 a patient determines that their aligner does not fit. The aligner fit photo may be captured by a patient device and may be sent, via an application on the patient's device to a treatment system device where the image an analyzed to determine the extent of the aligner's fit or lack thereof.

Aligner fit analysis may include receiving image data of a patient's dentition and an orthodontic appliance. For example, the treatment system device may receive image data image data of the patient's dentition. As described herein, the patient may take their own photographs of their own dentition using their own devices. This image data may include image data captured with the patient wearing their orthodontic appliance, which may be a clear aligner. The patient may capture the image data during a middle or near an end of a treatment stage, although the patient may capture the image data at any time.

The systems described herein may perform this process in a variety of ways. In one example, the image data may be uploaded from a patient's device to another computing device, such as a server or other computer, such as the treatment system device for further processing. In other examples, the image data may be processed on the patient's device.

One or more of the systems described herein may identify, from the image data, the orthodontic appliance. For example, the treatment system device may identify the orthodontic appliance, which may be a clear aligner.

The treatment system device may identify the orthodontic appliance in a variety of ways. In one example, semantic segmentation may be performed to classify each pixel of the image data into one of a plurality of classes. For example, a probability of belonging to each class may be determined for each pixel of the image data. Each pixel may be classified based on which class the pixel has the highest probability of matching. The classes may include, for example, a tooth class indicating the patient's teeth (which may be portions of the teeth not covered by the orthodontic appliance), a gap class indicating a gap between the orthodontic appliance and a corresponding gingival edge, and a space class indicating a space between an incisal edge of the orthodontic appliance and an incisal edge of a corresponding tooth. In other examples, other classes may be used, such as a gum class corresponding to the patient's gums, an appliance class, other classes, etc. By performing the semantic segmentation, pixels corresponding to the orthodontic appliance (e.g., the gap class and the space class) may be distinguished from pixels corresponding to the patient's dentition without the appliance (e.g., the tooth class). As will be described further below, the gap class and/or the space class may also correspond to a misalignment.

Mask data in which semantic segmentation has identified a gap region and a space region between the patient's dentition and the aligner.

In some examples, the semantic segmentation may be performed using machine learning. For example, a neural network or other machine learning scheme may be used to perform the semantic segmentation. In some example, the neural network may be trained to perform the semantic segmentation by inputting an image data set, such as a training data set, for semantic segmentation by the neural network. This training data set may have a corresponding mask data set of the desired semantic segmentation. The training may further include computing an error between an output of the neural network (e.g. by performing the semantic segmentation) and the mask data set corresponding to the image data set, and adjusting the parameters of the neural network to reduce the error.

In other examples, identifying the orthodontic appliance may include evaluating a color value of each pixel to identify a tooth portion without the orthodontic appliance and a tooth portion with the orthodontic appliance. For instance, a threshold-based segmentation may be used in which color thresholds corresponding to teeth, gums, appliances over teeth, and appliances without teeth, may be used to classify each pixel.

In other examples, identifying the orthodontic appliance may include applying one or more filters to the image data to determine a tooth edge and an orthodontic appliance edge. For instance, an edge-based segmentation may be used to find edges and regions inside the edges may be designated by class based on color features, such as the color threshold described herein.

In some examples, the various segmentation schemes described herein may be applied per tooth such that different segmentation schemes may be applied to different identified teeth. By identifying tooth-to-tooth boundaries, each tooth may be analyzed to provide tooth-specific information or data. For example, color evaluation may be applied per tooth such that color values and/or thresholds may be local to each tooth. Differences in lighting and/or actual differences between tooth colors may affect global color values whereas local tooth color analysis may more readily identify between classes. In another example, semantic segmentation may be applied to identify spaces per tooth. The semantic segmentation scheme may use a semantic segmentation model to find spacing for a given tooth, such as upper-left central incisor, etc. Alternatively, each tooth may be identified in the image data and identified tooth spacing may be associated to the corresponding specific tooth.

One or more of the systems described herein may calculate a misalignment height of a misalignment of the orthodontic appliance with respect to the patient's dentition. For example, the treatment system device may calculate the misalignment height of a misalignment determined using the identified orthodontic appliance.

The treatment system may calculate the misalignment height of a misalignment in a variety of ways. In one example, the misalignment height may be calculated from a pixel height of the misalignment, which may be identified from misalignment classes such as the gap class and/or the space class as described herein.

Each misalignment may occur in several regions, such as across a horizontal range. In such examples, the misalignment dimension (e.g., height, length, and/or width) may be calculated from aggregating the plurality of identified misalignments. For example, for space region, the various heights across space region may be determined. The misalignment height for space region may be calculated using, for example, an 80th percentile value of the various heights, although in other examples, other percentiles may be used such that outlier values may not significantly impact the misalignment height. Alternatively, other aggregating functions, such as average, mode, etc. may be used. The misalignment height for gap region and space region may be similarly calculated.

Although pixel heights may be used, in some examples, the pixel height may be converted to a standard unit of measurement. For instance, the patient's doctor may prefer to see misalignment heights measured in millimeters or other unit of measurement. To convert the pixel measurement, a reference object, which may be an identifiable subset of teeth such as an incisor, may be identified from the image data. The reference object may be selected based on having an available known measurement. For example, the incisor measurement may be obtained from a treatment plan for the patient. A pixel height of the incisor may be determined from the image data (for example by determining edges for the identified incisor and counting pixels along a desired dimension) and used with the incisor measurement to determine a conversion factor between pixels and the standard unit of measurement (e.g., mm). The misalignment height may be converted from pixels to the standard unit of measurement using the conversion factor.

In some other examples, the conversion factor may be determined using a global average of pixels-per-tooth of all identified teeth, optionally excluding outlier values. In yet other examples, the conversion factor may be determined by constructing a field of pixel-to-mm sizes over an entirety of the image data and interpolating and/or extrapolating pixel-to-mm sizes across the identified arch.

In some examples, the misalignment height may be further adjusted. The semantic segmentation may overestimate misalignment regions. In such instances, a thickness offset may be subtracted from the calculated misalignment height to simulate a material thickness of the orthodontic appliance. The thickness offset may be obtained from a treatment plan for the patient.

In some examples, the misalignment height may be tracked over time using image data over time. For example, the patient may capture image data at various points in time during a treatment stage. A misalignment trend may be identified from the tracked misalignment heights. The misalignment trend may be defined as a general trend (e.g., increasing, decreasing, etc.), as height deltas (e.g., the changes in misalignment height at each point in time), or by actual misalignment height values.

One or more of the systems described herein may determine whether the misalignment height satisfies a misalignment threshold. For example, the treatment system device may determine whether the misalignment height satisfies a misalignment threshold. The misalignment threshold may be predetermined or precalculated, such as based on patient history and/or other empirical data, or may be manually selected, such as by the patient's doctor.

In some embodiments, the misalignment threshold may comprise a plurality of misalignment thresholds. For example, 0.5 mm space may not be desirable but may not necessarily require corrective action and therefore may be set as a low threshold. However, 0.75 mm may require corrective action and thus be set as a high threshold. In some examples, if the misalignment trend is tracked, the misalignment threshold may include a misalignment trend threshold. For example, if the misalignment height remains at 0.75 mm at multiple points of time, corrective action may be needed.

One or more of the systems described herein may, in response to satisfying the misalignment threshold, provide a notification. For example, a notification may be provided to the doctor device if the misalignment exceeds the threshold.

In some embodiments, the notification may include a message or other notification to the patient's doctor. In some examples, the notification may include providing a visual overlay of the misalignment. In some examples, a color may indicate a type of misalignment.

In some examples, if the misalignment threshold includes a plurality of misalignment thresholds, the notification may include increasing priority based on the threshold met. For each range between the multiple thresholds, a different color may be used when depicting mask data. For example, if the misalignment height is below a low threshold, a low priority color such as blue may be used. If between the low and high threshold, a low warning color such as yellow may be used. If exceeding the high threshold, a high warning color such as orange may be used.

In some examples, the misalignment threshold may include the misalignment trend threshold. The notification may be provided in response to satisfying the misalignment trend threshold.

At block 1515 the doctor may review the aligner fit analysis and threshold information and determine whether or not the patient should have their teeth rescanned. If there is a determination that the teeth should be rescanned, then the doctor may send a rescan inquiry or request to the patient.

At block 1525, the treatment system may receive an aligner fit determination from the doctor. In some embodiments, at block 1525, the treatment system may receive a request form the doctor system to request or inquire of the patient on how they want to rescan their teeth. For example, the treatment system may send a request or inquiry to the patient via the patient device to inquire if the patient wants to go into the doctor's office to rescan their teeth or if they want to use a home rescan process, such as through the use of impressionable material or home scanning discussed herein.

At block 1530, the patient chooses a rescan option and the patient device sends the choice to the treatment system device.

At block 1535, the treatment system receives a request for home rescan or receives the chosen option, such as for home rescan. The treatment system may then query a patient database that contains information about the patient, such as their address. The treatment system may also retrieve treatment information from, such as the shape and size, such as width and length, of the patient's dentition, based on initial scan data. From the treatment system may determine a size of impressionable material to send to the patient at the patient's address from a plurality of sizes.

At block 1540, the patient generates or takes an impression. Block 1540 may carry out one or more of blocks 1010, 1020, 1030, 1110, 1120, and 1130. In some embodiments, the impression may be sent to a treatment provider. In some embodiments, the impression may be scanned by the patient using a patient device, which may be a smartphone that includes a camera. The scanning process may include any of the processes discussed herein, such as those shown and described with respect to FIGS. 10, 11, 16, and 17 or other scanning methods.

In some embodiments, at block 1540 the patient deice may guide the user through the process of capturing images, such as by providing instructions on how many and at which angles and positions the images should be captured. The device may provide feedback based on the captured images, indicating that additional image should be taken, or by highlighting areas of the 3D model of the impression that are missing or need additional data and that should be captured in additional images. In some embodiments, the image may be processed on the patient device to generate a 3D model of the patient's dentition. In some embodiments, the images may be sent to the treatment system device and/or the doctor device for processing into a 3D model of the current state of the patient's dentition.

In some embodiments, the process may start at block 1540 or 1535. For example, an impression kit, which may include a bite block and/or an apparatus to aid in capturing images of a bite block impression, such as apparatus 1704 may be sent to the patient or the patient may have multiple bite blocks for use in taking impressions as part of their treatment. In some embodiments, the patient device may notify the patient to take a bite impression and/or scan the bite impression based on a treatment plan, such as with on at specific times, such as every month or every three months. The notifications may be based on treatment stages, such as taking impressions every third, fourth, fifth, or sixth stage.

At block 1545, the treatment system may update the treatment plan for the patient. Block 1545 may carry out one or more of blocks 1020, 1030, 1040, 1050, 1060, 1120, 1130, 1140, 1150, and 1160. The updated treatment plan or updated aligner model may be sent to a doctor for review, revisions/feedback, and approval.

Block 1550 the doctor system may receive revisions, feedback, or approval of the treatment plan or retainer. In some embodiments, the revisions may include revisions to tooth positions in one or more intermediate and/or the final stage. The revisions may be sent to and received by the treatment system which may generate further updates to the treatment or retainer and send the revised treatment or retainer to the doctor system for further review and/or approval. This may be an iterative processes. Once the doctor is satisfied, the doctor system may send the approval of the treatment plan or retainer to the treatment system.

At block 1555 the doctor system may send and the treatment system may receive delivery instructions, such as to deliver the updated retainer or aligners directly to the patient or to the doctor for the doctor to deliver to the patient.

At block 1560, the shipping information, such as address, may be determined by retrieving the patient or doctor address from a database.

At block 1565, the aligners or retainer for treating the patient are fabricated and shipped to the patient or doctor, for delivery to the patient. At block 1570, the patient receives the aligners or retainer. At block 1570, a progress photo may be taken to confirm aligner or retainer fit, such as discussed with respect to block 1505.

At block 1575, aligner fit analysis may be carried out, such as described with respect to block 1510.

While the foregoing disclosure sets forth various embodiments using specific block diagrams, flowcharts, and examples, each block diagram component, flowchart step, operation, and/or component described and/or illustrated herein may be implemented, individually and/or collectively, using a wide range of hardware, software, or firmware (or any combination thereof) configurations. In addition, any disclosure of components contained within other components should be considered example in nature since many other architectures can be implemented to achieve the same functionality.

In some examples, all or a portion of the example systems disclosed herein may represent portions of a cloud-computing or network-based environment. Cloud-computing environments may provide various services and applications via the Internet. These cloud-based services (e.g., software as a service, platform as a service, infrastructure as a service, etc.) may be accessible through a web browser or other remote interface. Various functions described herein may be provided through a remote desktop environment or any other cloud-based computing environment.

In various embodiments, all or a portion of example systems disclosed herein may facilitate multi-tenancy within a cloud-based computing environment. In other words, the software modules described herein may configure a computing system (e.g., a server) to facilitate multi-tenancy for one or more of the functions described herein. For example, one or more of the software modules described herein may program a server to enable two or more clients (e.g., customers) to share an application that is running on the server. A server programmed in this manner may share an application, operating system, processing system, and/or storage system among multiple customers (i.e., tenants). One or more of the modules described herein may also partition data and/or configuration information of a multi-tenant application for each customer such that one customer cannot access data and/or configuration information of another customer.

According to various embodiments, all or a portion of example systems disclosed herein may be implemented within a virtual environment. For example, the modules and/or data described herein may reside and/or execute within a virtual machine. As used herein, the term “virtual machine” generally refers to any operating system environment that is abstracted from computing hardware by a virtual machine manager (e.g., a hypervisor). Additionally or alternatively, the modules and/or data described herein may reside and/or execute within a virtualization layer. As used herein, the term “virtualization layer” generally refers to any data layer and/or application layer that overlays and/or is abstracted from an operating system environment. A virtualization layer may be managed by a software virtualization solution (e.g., a file system filter) that presents the virtualization layer as though it were part of an underlying base operating system. For example, a software virtualization solution may redirect calls that are initially directed to locations within a base file system and/or registry to locations within a virtualization layer.

In some examples, all or a portion of example systems disclosed herein may represent portions of a mobile computing environment. Mobile computing environments may be implemented by a wide range of mobile computing devices, including mobile phones, tablet computers, e-book readers, personal digital assistants, wearable computing devices (e.g., computing devices with a head-mounted display, smartwatches, etc.), and the like. In some examples, mobile computing environments may have one or more distinct features, including, for example, reliance on battery power, presenting only one foreground application at any given time, remote management features, touchscreen features, location and movement data (e.g., provided by Global Positioning Systems, gyroscopes, accelerometers, etc.), restricted platforms that restrict modifications to system-level configurations and/or that limit the ability of third-party software to inspect the behavior of other applications, controls to restrict the installation of applications (e.g., to only originate from approved application stores), etc. Various functions described herein may be provided for a mobile computing environment and/or may interact with a mobile computing environment.

In addition, all or a portion of example systems disclosed herein may represent portions of, interact with, consume data produced by, and/or produce data consumed by one or more systems for information management. As used herein, the term “information management” may refer to the protection, organization, and/or storage of data. Examples of systems for information management may include, without limitation, storage systems, backup systems, archival systems, replication systems, high availability systems, data search systems, virtualization systems, and the like.

In some embodiments, all or a portion of example systems disclosed herein may represent portions of, produce data protected by, and/or communicate with one or more systems for information security. As used herein, the term “information security” may refer to the control of access to protected data. Examples of systems for information security may include, without limitation, systems providing managed security services, data loss prevention systems, identity authentication systems, access control systems, encryption systems, policy compliance systems, intrusion detection and prevention systems, electronic discovery systems, and the like.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

Unless otherwise noted, the terms “connected to” and “coupled to” (and their derivatives), as used in the specification and claims, are to be construed as permitting both direct and indirect (i.e., via other elements or components) connection. In addition, the terms “a” or “an,” as used in the specification and claims, are to be construed as meaning “at least one of.” Finally, for ease of use, the terms “including” and “having” (and their derivatives), as used in the specification and claims, are interchangeable with and shall have the same meaning as the word “comprising.

The processor as disclosed herein can be configured with instructions to perform any one or more steps of any method as disclosed herein.

It will be understood that although the terms “first,” “second,” “third”, etc. may be used herein to describe various layers, elements, components, regions or sections without referring to any particular order or sequence of events. These terms are merely used to distinguish one layer, element, component, region or section from another layer, element, component, region or section. A first layer, element, component, region or section as described herein could be referred to as a second layer, element, component, region or section without departing from the teachings of the present disclosure.

As used herein, the term “or” is used inclusively to refer items in the alternative and in combination.

As used herein, characters such as numerals refer to like elements.

Embodiments of the present disclosure have been shown and described as set forth herein and are provided by way of example only. One of ordinary skill in the art will recognize numerous adaptations, changes, variations and substitutions without departing from the scope of the present disclosure. Several alternatives and combinations of the embodiments disclosed herein may be utilized without departing from the scope of the present disclosure and the inventions disclosed herein. Therefore, the scope of the presently disclosed inventions shall be defined solely by the scope of the appended claims and the equivalents thereof.

1. A system for remote progress tracking of orthodontic treatment, the system comprising:
a processor; and
memory comprising instructions that when executed by the processor cause the system to:
receive a 3D digital model of a patient's dentition including the patient's teeth;
generate a 3D digital model of an impression of occlusal surfaces of the patient's teeth;
register the teeth of the 3D digital model of the patient's dentition to the position and orientation of the patient's teeth in the 3D digital model of the impression of the occlusal surfaces of the patient's teeth; and
generate an updated 3D digital model of the patient's teeth based on the registration.

2. The system of claim 1, wherein the instruction to generate the 3D digital model include instructions to:
receive a plurality of 2D images of an impression of the occlusal surface of the patient's teeth, and wherein the 3D digital model of an impression of occlusal surfaces of the patient's teeth is generated from the plurality of 2D images of the impression.

3. The system of claim 2, wherein the instructions further comprise instruction to generate an updated treatment plan to move the patient's teeth from an arrangement of the teeth in the updated 3D digital model towards a final arrangement.

4. The system of claim 2, wherein the instructions to receive a 3D digital model of the impression of the occlusal surfaces of the patient's teeth include instructions to generate the 3D digital model of the impression of the occlusal surfaces of the patient's teeth.

5. The system of claim 1, wherein the impression is patterned.

6. The system of claim 1, wherein the instructions to register include instructions to match the surface features of the teeth of the 3D digital model of the patient's dentition to the surface features of the patient's teeth in the 3D digital model of the impression of the occlusal surfaces of the patient's teeth.

7. The system of claim 1, wherein the impression includes less than 10% of the height of the crown of the patient's teeth.

8. The system of claim 1, wherein the impression includes the cusps and grooves of the occlusal surface of the patient's dentition.

9. The system of claim 1, wherein the impression includes between 2 mm and 4 mm of the height of the crown of the patient's teeth.

10. The system of claim 1, wherein the 3D digital model of a patient's dentition including the patient's teeth is a segmented model.

11.-30. (canceled)

31. A method of remote progress tracking of orthodontic treatment, the method comprising:
receiving a 3D digital model of a patient's dentition including the patient's teeth;
generating a 3D digital model of an impression of occlusal surfaces of the patient's teeth;
registering the teeth of the 3D digital model of the patient's dentition to the position and orientation of the patient's teeth in the 3D digital model of the impression of the occlusal surfaces of the patient's teeth; and
generating an updated 3D digital model of the patient's teeth based on the registration.

32. The method of claim 30, further comprising generating an updated treatment plan to move the patient's teeth from an arrangement of the teeth in the updated 3D digital model towards a final arrangement.

33. The method of claim 30, wherein receiving a 3D digital model of the impression of the occlusal surfaces of the patient's teeth includes generating the 3D digital model of the impression of the occlusal surfaces of the patient's teeth.

34. The method of claim 32, wherein generating the 3D digital model of the impression of the occlusal surfaces of the patient's teeth includes scanning the physical bite block impression.

35. The method of claim 33, wherein the bite block impression is patterned.

36. The method of claim 30, wherein registering includes matching the surface features of the teeth of the 3D digital model of the patient's dentition to the surface features of the patient's teeth in the 3D digital model of the impression of the occlusal surfaces of the patient's teeth.

37. The method of claim 30, wherein the impression includes less than 10% of the height of the crown of the patient's teeth.

38. The method of claim 30, wherein the impression includes the cusps and grooves of the occlusal surface of the patient's dentition.

39. The method of claim 30, wherein the impression includes between 2 mm and 4 mm of the height of the crown of the patient's teeth.

40. The method of claim 30, wherein the 3D digital model of a patient's dentition including the patient's teeth is a segmented model.

41.-60. (canceled)
